(19) United States
(12) Patent Application Publication
(10) Pub. No.: US 2017/0080195 A1
(43) Pub. Date: Mar. 23, 2017

(54) EXPANSION DEVICE

(71) Applicant: TERUMO KABUSHIKI KAISHA, Tokyo (JP)

(72) Inventor: Takeshi DOSONO, Shizuoka (JP)

(73) Assignee: TERUMO KABUSHIKI KAISHA, Tokyo (JP)

(21) Appl. No.: 15/368,264

(22) Filed: Dec. 2, 2016

Related U.S. Application Data
(63) Continuation of application No. PCT/JP2015/065559, filed on May 29, 2015.

(30) Foreign Application Priority Data
Jun. 3, 2014 (JP) 2014-114533
Jan. 13, 2015 (JP) 2015-003865

(51) Int. Cl. A61M 29/02; A61F 2/958

(52) U.S. Cl. CPC A61M 29/02; A61F 2/958

(57) ABSTRACT

An expansion device is disclosed, which includes an outer tube in which a plurality of wire portions extending along an axial direction are disposed so as to be arranged in a circumferential direction in the vicinity of a distal portion, and an inner tube that is inserted into the outer tube and is interlocked with the distal portion of the outer tube. The wire portion has an expansion portion which expands radially outward when the outer tube is compressed in the axial direction, a distal side fragile portion which is formed on the distal side of the expansion portion and has flexural rigidity lower than that of the expansion portion, and a proximal side fragile portion which is formed on the proximal side of the expansion portion and having flexural rigidity lower than that of the expansion portion.

### CROSS-REFERENCES TO RELATED APPLICATIONS

This application is a continuation of International Application No. PCT/JP2015/065559 filed on May 29, 2015, which claims priority to Japanese Application No. 2014-114533 filed on Jun. 3, 2014, and Japanese Application No. 2015-003865 filed on Jan. 13, 2015, the entire contents of which are incorporated herein by reference.

### TECHNICAL FIELD

The present disclosure relates to an expansion device having an expansion portion which expands inside a biological lumen.

### BACKGROUND DISCUSSION

Recently, for example, in treatment of myocardial infarction and angina pectoris, a method in which a stent is caused to indwell at a lesion area (stenosed site) in the coronary artery so as to ensure a space inside the coronary artery has been performed. A similar method has been sometimes performed so as to improve a stenosed site generated inside a different blood vessel, the bile duct, the trachea, the esophagus, the urethra, and other biological lumens.

As one of the methods in which the stent expands inside a biological lumen, there is a generally known method of adopting a balloon catheter. The balloon catheter is provided with a balloon which can be dilated when a fluid is supplied, and a stent having a tubular shape in its entirety and being formed with wires is disposed on the balloon in a deflated state. When the balloon is dilated at a target position, the wires of the stent are plastically deformed and the stent is increased in diameter. Accordingly, the stent can be tightly fixed inside a biological lumen.

However, for example, in a case where the balloon catheter is used inside a blood vessel, when the balloon is dilated, the blood vessel is blocked by the balloon. Accordingly, the blood flow is hindered and a burden to a living body can increase.

Therefore, for example, in JP-A-2005-270394, a treatment tool provided with an expansion portion which can expand inside a biological lumen without hindering the blood flow is disclosed. The treatment tool is provided with a tube in which a plurality of slits extending in an axial direction is formed in a circumferential direction, and a core that is inserted into the tube and is fixedly attached to a distal portion of the tube. The treatment tool has a structure in which the core compresses the tube in the axial direction and a wire-like portion (expansion portion) formed between the slits adjacent to each other is deformed and expands so as to protrude radially outward from the tube. According to the treatment tool, a gap is formed between the wire-like portions configuring the expansion portions. Therefore, a burden to a living body can be reduced without hindering a blood flow.

When a treatment instrument disclosed in JP-A-2005-270394 is adopted, only a narrow range of a central portion in an expansion portion of a tube expands so as to significantly protrude radially outward. Therefore, pressing can be performed with only the central portion in the expansion portion, and thus, a wide range cannot be uniformly pressed. In addition, in a case where a wall surface of a biological lumen is hardened, the expansion portion of the tube is out-pushed by the wall surface and is deformed, thereby being likely to have an uniformed shape. Moreover, since only the central portion in the expansion portion of the tube significantly protrudes, an excessive load can be applied to a portion pressed by the central portion, and thus, a load applied to a living body increases.

### SUMMARY

An expansion device is disclosed in which a load applied to a living body can be reduced and a wide range of an expansion portion can uniformly expand.

An expansion device is disclosed, which includes a pipe body in which a plurality of wire portions extending along an axial direction are disposed so as to be arranged in a circumferential direction in the vicinity of a distal portion, and a core that is inserted into the pipe body and is interlocked with the distal portion of the pipe body. The wire portion has an expansion portion which expands radially outward when the pipe body is compressed in the axial direction, a distal side fragile portion which is formed on a distal side of the expansion portion and has flexural rigidity lower than that of the expansion portion, and a proximal side fragile portion which is formed on a proximal side of the expansion portion and has flexural rigidity lower than that of the expansion portion.

In the expansion device having the above-described configuration, the distal side fragile portion having flexural rigidity lower than that of the expansion portion is formed on the distal side of the expansion portion, and the proximal side fragile portion having flexural rigidity lower than that of the expansion portion is formed on the proximal side of the expansion portion. Thus, in a case where compressive force acts on the pipe body, the distal side fragile portion and the proximal side fragile portion warp more significantly than the expansion portion, and the expansion portion expands radially outward in a substantially uniform manner. Therefore, a load applied to a living body by the expansion portion can be reduced, and a wide range of the expansion portion can uniformly expand.

The distal side fragile portion and the proximal side fragile portion may be set to have flexural rigidity lower than that of the expansion portion due to at least one of the material, the width in the circumferential direction, and the thickness different from that of the expansion portion. Accordingly, the flexural rigidity of the distal side fragile portion and the proximal side fragile portion can be set relatively easily and suitably.

In the pipe body, a main slit may be formed between the expansion portions adjacent to each other in the circumferential direction, a plurality of bifurcated slits bifurcated from at least one of the distal side and the proximal side of the main slit may be formed, and the width of at least one of the distal side fragile portion and the proximal side fragile portion in the circumferential direction may be set to be smaller than that of the expansion portion due to the provided bifurcated slits. Accordingly, the flexural rigidity of the expansion portion, the distal side fragile portion, and the proximal side fragile portion can be set relatively easily and suitably by only forming the main slit and the bifurcated slit.

The pipe body may have a first pipe portion in which a plurality of first slits are formed with space therebetween in the circumferential direction, and a second pipe portion which is disposed between the first pipe portion and the core and in which a plurality of second slits are formed with space therebetween in the circumferential direction. The expansion portions may include a plurality of first expansion portions which are formed between the first slits in the first pipe portion, and a plurality of second expansion portions which are formed between the second slits in the second pipe portion. The plurality of first slits and the plurality of second slits may be formed at positions different from each other in the circumferential direction. Accordingly, the expansion portions expand in response to compressive force acting in the axial direction of the pipe body due to relative movement between the pipe body and the core in the axial direction. Thus, an expansion target can be caused to expand without adopting a dilation fluid. In addition, a biological lumen is not completely occluded when the expansion portions expand. Thus, the expansion target can be caused to expand without, interrupting a flow of a body fluid such as blood. In addition, the expansion portion has a dual structure having the first expansion portions on the outer side and the second expansion portions on the inner side, and the plurality of first slits and the plurality of second slits are formed at positions different from each other in the circumferential direction. Thus, the expansion target can be pressed from the inside over a wide range in the circumferential direction. Accordingly, the expansion target can be uniformly widened in the circumferential direction.

The first expansion portions and the second expansion portions may be alternately arranged in the circumferential direction while the expansion portions are in an expansion state. Accordingly, the expansion target can be more uniformly widened in the circumferential direction.

The expansion portion may have a restriction portion that restricts the second expansion portion from protruding outward beyond the first expansion portion by being disposed between the first expansion portions adjacent to each other in the circumferential direction and outside the second expansion portion while the first expansion portions are in an expansion state. Accordingly, the second expansion portion can be prevented from protruding outward beyond the first expansion portion. Thus, the second expansion portion can be prevented from failing to contract again.

The restriction portion may be configured to be formed of a film member which is folded while the expansion portions are in a contraction state and is deployed in response to expansion of the space between the first expansion portions adjacent to each other in the circumferential direction when the expansion portions expand. Accordingly, the restriction portion can be realized through a relatively simple configuration.

The film member may extend along the first slit. Accordingly, a gap between the first expansion portions adjacent to each other in the circumferential direction is eliminated over a region elongated in the axial direction. Thus, the expansion target can be more uniformly widened in the circumferential direction.

The film member may be interposed between the first expansion portion and the second expansion portion while the expansion portions are in a contraction state. Accordingly, the film member is folded and stored inside the expansion portion. Thus, the expansion portion can be effectively suppressed from being increased in diameter due to the provided film member.

The first pipe portion may have a plurality of first wire portions which are disposed so as to be arranged in the circumferential direction. The second pipe portion may have a plurality of second wire portions which are disposed so as to be arranged in the circumferential direction. The first wire portion may have the first expansion portion which expands radially outward when the first pipe portion is compressed in the axial direction, a first distal side fragile portion which is formed on the distal side of the first expansion portion and has flexural rigidity lower than that of the first expansion portion, and a first proximal side fragile portion which is formed on the proximal side of the first expansion portion and has flexural rigidity lower than that of the first expansion portion. The second wire portion may have the second expansion portion which expands radially outward when the second pipe portion is compressed in the axial direction, a second distal side fragile portion which is formed on the distal side of the second expansion portion and has flexural rigidity lower than that of the second expansion portion, and a second proximal side fragile portion which is formed on the proximal side of the second expansion portion and has flexural rigidity lower than that of the second expansion portion. Accordingly, the first distal side fragile portion and the first proximal side fragile portion warp more significantly than the first expansion portion in response to compressive force acting in the axial direction of the first pipe portion. Thus, the first expansion portion expands radially outward in a substantially uniform manner in the axial direction. In addition, the second distal side fragile portion and the second proximal side fragile portion warp more significantly than the second expansion portion in response to compressive force acting in the axial direction of the second pipe portion. Thus, the second expansion portion expands radially outward in a substantially uniform manner in the axial direction. Therefore, a wide range of the expansion portion in the axial direction can uniformly expand, and a load applied to a living body by the expansion portion can be further reduced.

The second distal side fragile portion may have a length in the axial direction longer than that of the first distal side fragile portion. The second proximal side fragile portion may have a length in the axial direction longer than that of the first proximal side fragile portion. Accordingly, while the expansion portions are in an expansion state, the outer diameter formed with the plurality of first expansion portions and the outer diameter formed with the plurality of second expansion portions can be substantially equal to each other. Thus, the expansion portions can expand in a substantially uniform manner in the circumferential direction.

The expansion portions may include an X-ray contrast material. Accordingly, position of the expansion portion positioned inside a biological lumen can be checked, and the expansion portion can expand at the exact position.

The core may include an X-ray contrast material in a portion positioned on the inner side of the expansion portions. Accordingly, the position of the expansion portion positioned inside a biological lumen can be checked, and the expansion portion can expand at the exact position.

A method is disclosed of treating a blood vessel, the method comprising: positioning an expansion member within the blood vessel, the expansion member including a pipe body in which a plurality of wire portions extending along an axial direction are disposed so as to be arranged in a circumferential direction in a distal portion, and a core that is inserted into the pipe body and is interlocked with the distal portion of the pipe body, wherein the wire portion has an expansion portion which expands radially outward when the pipe body is compressed in the axial direction, a distal side fragile portion which is formed on a distal side of the expansion portion and has flexural rigidity lower than that of the expansion portion, and a proximal side fragile portion which is formed on a proximal side of the expansion portion and has flexural rigidity lower than that of the expansion portion; and radially expanding the expansion portion within the blood vessel.

### BRIEF DESCRIPTION OF THE DRAWINGS

FIG. 1 is a plan view illustrating an expansion device, according to a first embodiment.

FIG. 2 is a plan view illustrating a distal portion of the expansion device, according to the first embodiment.

FIG. 3 is a cross-sectional view illustrating the distal portion of the expansion device, according to the first embodiment.

FIG. 4 is a cross-sectional view illustrating the distal portion of a modification example of the expansion device, according to the first embodiment.

FIG. 5 is a cross-sectional view illustrating the distal portion of another modification example of the expansion device, according to the first embodiment.

FIG. 6 is a plan view illustrating the distal portion of further another modification example of the expansion device, according to the first embodiment.

FIG. 7 is a plan view illustrating a state where an expansion portion of the expansion device expands, according to the first embodiment.

FIG. 8 is a plan view illustrating the distal portion when the expansion portion of the expansion device expands, according to the first embodiment.

FIG. 9 is a cross-sectional view illustrating the distal portion when the expansion portion of the expansion device expands, according to the first embodiment.

FIG. 10 is a plan view illustrating the distal portion of an expansion device, according to a second embodiment.

FIG. 11 is a plan view illustrating a state where the expansion portion of the expansion device expands, according to the second embodiment.

FIG. 12 is a perspective view illustrating an expansion device, according to a third embodiment.

FIG. 13 is a plan view illustrating an expansion mechanism and a peripheral portion thereof in the expansion device, according to the third embodiment.

FIG. 14 is a cross-sectional view illustrating the expansion mechanism and the peripheral portion thereof in the expansion device, according to the third embodiment.

FIG. 15 is a cross-sectional view of the expansion mechanism in a contraction state.

FIG. 16 is a cross-sectional view of the expansion mechanism in an expansion state.

FIG. 17 is a plan view illustrating the expansion mechanism and the peripheral portion of the expansion mechanism in an expansion state.

FIG. 18 is a plan view of the expansion mechanism and the peripheral portion of the expansion mechanism in an expansion device in a contraction state according to a fourth embodiment.

FIG. 19 is a cross-sectional view of the expansion mechanism of the expansion device in a contraction state according to the fourth embodiment.

FIG. 20 is a cross-sectional view of the expansion mechanism of the expansion device in an expansion state according to the fourth embodiment.

FIG. 21 is a cross-sectional view of the expansion mechanism of an expansion device in a contraction state according to a fifth embodiment.

FIG. 22 is a cross-sectional view of the expansion mechanism of the expansion device in an expansion state according to the fifth embodiment.

FIG. 23 is a plan view illustrating a modification example of the expansion portion of the expansion device, according to the first embodiment.

FIG. 24 is a plan view illustrating a state in FIG. 23 where the expansion portion of the expansion device expands.

FIG. 25 is a plan view illustrating another modification example of the expansion portion of the expansion device, according to the first embodiment.

FIG. 26 is a plan view illustrating a state in FIG. 25 where the expansion portion of the expansion device expands.

FIG. 27 is a plan view illustrating a modification example of an operation unit.

### DETAILED DESCRIPTION

Hereinafter, with reference to the drawings, embodiments of the present disclosure will be described. Note that, for the convenience of description, there are cases where the dimensional ratios of the drawings are exaggerated and are different from the actual ratios.

According to an expansion device 1 according to a first embodiment of the present disclosure, a stent 6 is caused to indwell at a stenosed site, an occluded site, generated inside a blood vessel, the bile duct, the trachea, the esophagus, the urethra, or other biological lumens so as to maintain an open state of the lumen. Note that, in this specification, a side inserted into the lumen will be referred to as “distal end” or “distal side”. An operating hand side will be referred to as “proximal end” or “proximal side”.

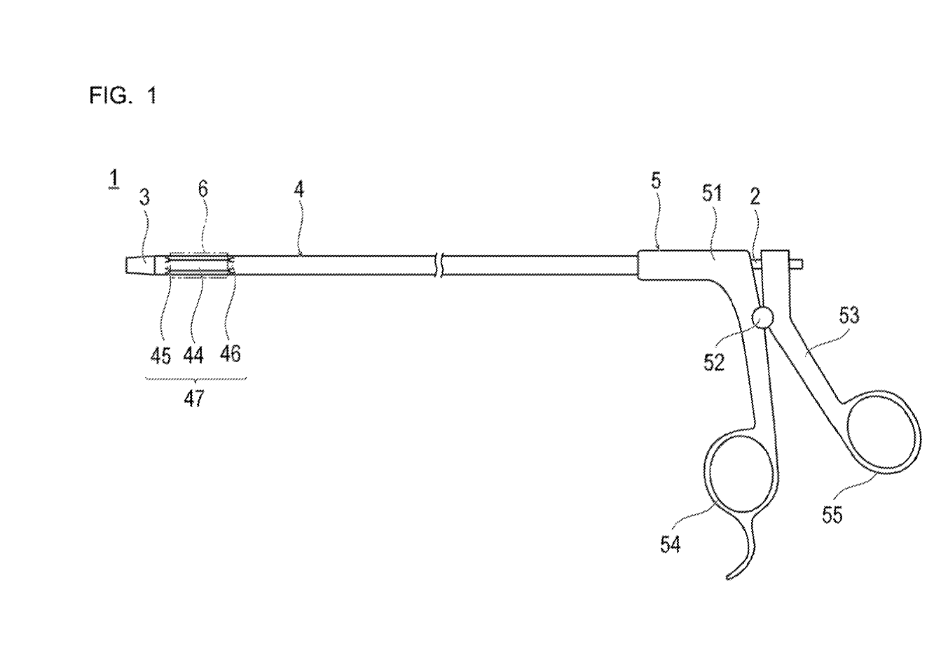

As illustrated in FIGS. 1 to 3, the expansion device 1 according to the first embodiment is provided with a tubular inner tube 2 (core), a distal member 3 which is fixedly attached to the outer circumferential surface of a distal portion of the inner tube 2, a tubular outer tube 4 (pipe body) covering the inner tube 2, and an operation unit 5 in which proximal portions of the inner tube 2 and the outer tube 4 are connected to each other.

In the inner tube 2, a guide wire lumen 21 allowing a guide wire to be inserted therein is formed. The distal member 3 is a member configuring the outermost distal portion of the expansion device 1 and can be fixedly attached to the outer circumferential surface of the distal portion of the inner tube 2. In order to reduce an influence to a living body when the expansion device 1 is inserted into a lumen, the distal member 3 is formed of a flexible material and is decreased in diameter toward the distal direction.

The outer tube 4 is a pipe body covering the inner tube 2 and can relatively move in the axial direction with respect to the inner tube 2. The distal portion of the outer tube 4 can be fixedly attached to the distal portion of the inner tube 2. Note that, the distal portion of the outer tube 4 may be fixedly attached to the inner tube 2 via the distal member 3. In addition, the distal member 3 may be a part of the inner tube 2 or a part of the outer tube 4.

In the vicinity of the distal end of the outer tube 4, a plurality of main slits 41 arranged in the circumferential direction are formed. Note that, the slit denotes a cut-off portion or an opening portion which extends linearly. Therefore, the slit may be an opening portion through which the inner tube 2 is exposed, in the present embodiment, there are provided four main slits 41, which are formed so as to have equal spaces among them in the circumferential direction and to be parallel to the axial line of the outer tube 4. In each of the main slits 41, a plurality of (two in the present embodiment) distal side bifurcated slits 42 (bifurcated slits) widened and bifurcated from the main slit 41 toward the distal direction are continuously formed. In addition, in each of the main slits 41, a plurality of (two in the present embodiment) proximal side bifurcated slits 43 (bifurcated slits) widened and bifurcated from the main slit 41 toward the proximal direction are continuously formed. Note that, the number of the main slits 41 is not limited as long as there are two or more main slits 41. In addition, the main slits 41 are not necessarily formed so as to have equal spaces among them in the circumferential direction. In addition, the main slits 41 may be formed so as to incline with respect to the axial line of the outer tube 4.

Between the main slits 41 adjacent to each other in the circumferential direction, an expansion portion 44 having a uniformly formed width in the circumferential direction is formed. The expansion portion 44 is a portion where the stent 6 is placed. On the distal side of the expansion portion 44, a distal side fragile portion 45 which continues from the expansion portion 44 is formed so as to be interposed between the distal side bifurcated slits 42. Since the distal side fragile portion 45 is formed so as to be interposed between the distal side bifurcated slits 42 widened and bifurcated from the main slit 41, the width in the circumferential direction is formed so as to be smaller than that of the expansion portion 44. In addition, on the proximal side of the expansion portion 44, a proximal side fragile portion 46 which continues from the expansion portion 44 is formed so as to be interposed between the proximal side bifurcated slits 43. Since the proximal side fragile portion 46 is formed so as to be interposed between the proximal side bifurcated slits 43 widened and bifurcated from the main slit 41, the width in the circumferential direction is formed so as to be smaller than that of the expansion portion 44. The distal side fragile portion 45, the expansion portion 44, and the proximal side fragile portion 46 form a wire portion 47 continuously extending along the axial line.

The distal side fragile portion 45, the expansion portion 44, and the proximal side fragile portion 46 can be formed so as to have a uniform thickness by using the same material. Therefore, the distal side fragile portion 45 and the proximal side fragile portion 46 having a width in the circumferential direction smaller than that of the expansion portion 44 have flexural rigidity lower than that of the expansion portion 44.

As the material forming the inner tube 2 and the outer tube 4, it is preferable that the material is hard and flexible. For example, it is possible to favorably use polyolefin such as polyethylene and polypropylene, polyamide, polyester such as polyethylene terephthalate, a fluorine-based polymer such as ETFE, polyether ether ketone (PEEK), polyimide, a shape memory alloy to which the shape memory effect or super-elasticity is applied through heat treatment, stainless steel, Ta, Ti, Pt, Au, or W. As the shape memory alloy, an Ni—Ti-based alloy, a Cu—Al—Ni-based alloy, or a Cu—Zn—Al-based alloy is preferably used.

The portion of the outer tube 4 in the vicinity of the distal end where the expansion portion 44, the distal side fragile portion 45, and the proximal side fragile portion 46 are formed may be formed of a member different from that of the portion of the outer tube 4 on the proximal side.

The length of the expansion device 1 (length from the outermost distal portion where the distal member 3 is provided to the operation unit 5) is not particularly limited. For example, in a case where the expansion device 1 is adopted in expansion of the blood vessel stent 6, the length preferably ranges, for example, from 1,200 mm to 1,600 mm. The outer diameter of the outer tube 4 is not particularly limited. For example, in a case where the outer tube 4 is adopted in expansion of the blood vessel stent 6, and the like, the outer diameter, for example, preferably ranges from 0.5 mm to 2.0 mm. The thickness of the outer tube 4 is not particularly limited. For example, in a case where the outer tube 4 is adopted in expansion of the blood vessel stent 6, the thickness, for example, preferably ranges from 0.10 mm to 0.35 mm.

As the material forming the distal member 3, it is preferable to adopt a flexible material. For example, a synthetic resin elastomer such as an olefin-based elastomer (for example, a polyethylene elastomer and a polypropylene elastomer), a polyamide elastomer, a styrene-based elastomer (for example, a styrene-butadiene-styrene copolymer, a styrene-isoprene-styrene copolymer, and a styrene-ethylene-butylene-styrene copolymer), polyurethane, a urethane-based elastomer, and a fluorine resin-based elastomer; or rubbers including synthetic rubber such as urethane rubber, silicone rubber, and butadiene rubber, as well as natural rubber such as latex rubber can be used.

The expansion portion 44 may be formed so as to include an X-ray contrast material in its material. Accordingly, the position of the stent 6 can be accurately grasped under X-ray contrast condition, and thus, the technique is more easily performed. As the X-ray contrast material, for example, gold, platinum, a platinum-iridium alloy, silver, stainless steel, molybdenum, tungsten, tantalum, palladium, or an alloy thereof is favorable.

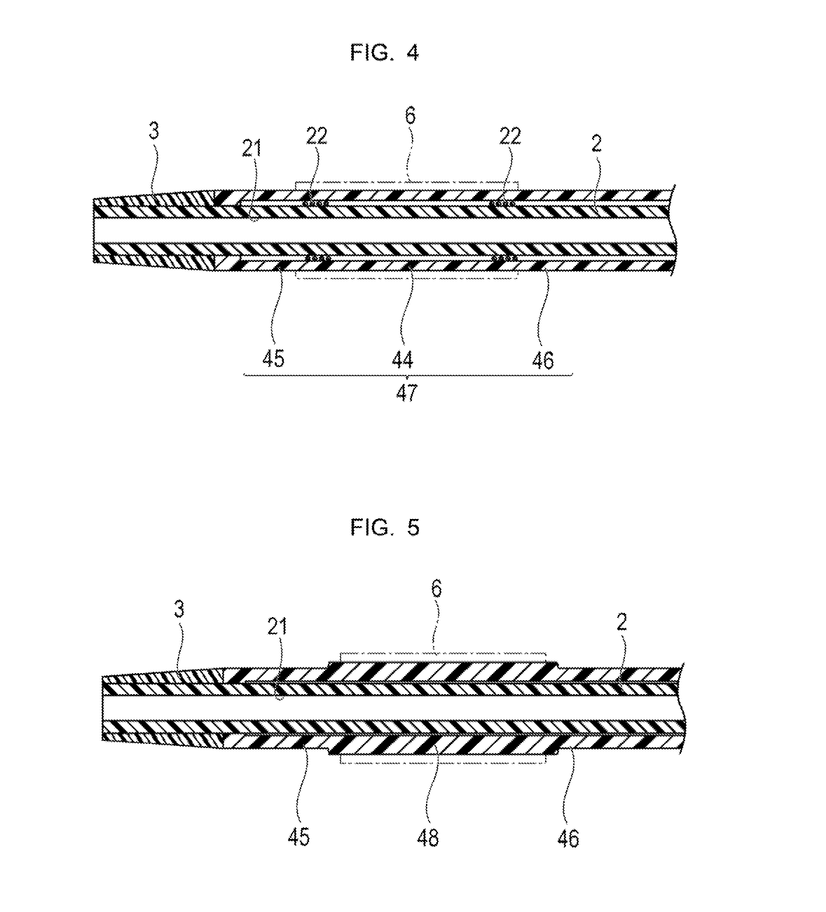

In addition, as in the modification example illustrated in FIG. 4, a marker 22 formed of an X-ray contrast material may be disposed at a position surrounded by the expansion portions 44 of the inner tube 2. The marker 22 is attached by winding a wire formed of an X-ray contrast material around the outer surface of the inner tube 2, or caulking or gluing a pipe formed of an X-ray contrast material onto the outer surface of the inner tube 2.

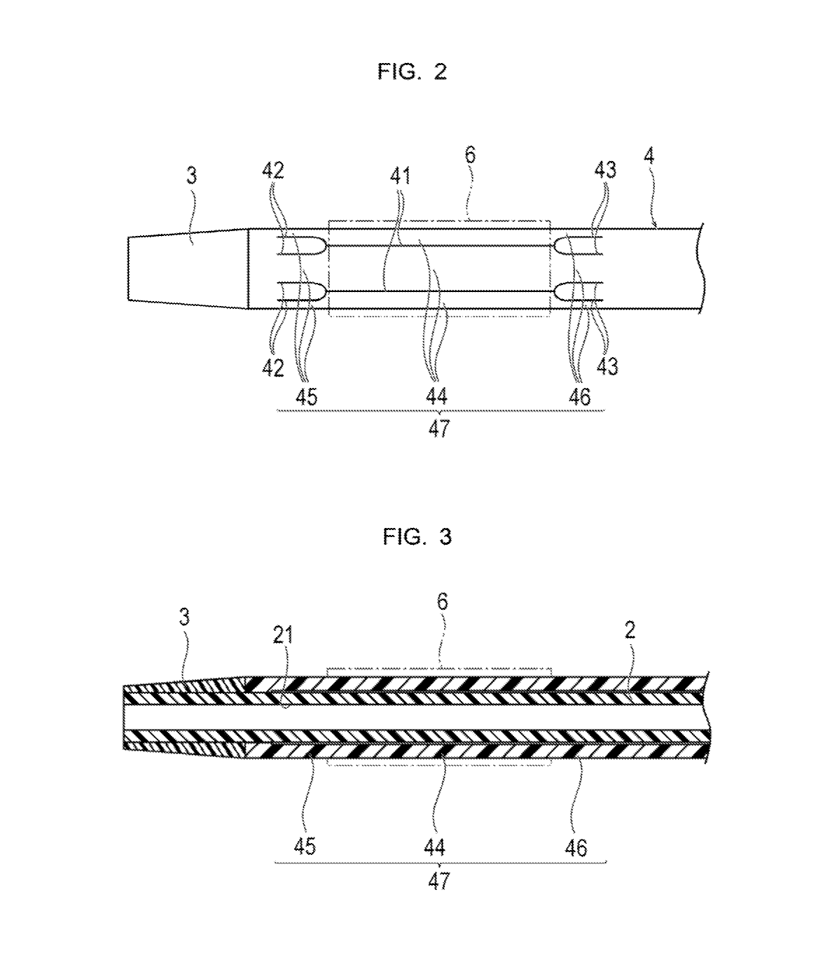

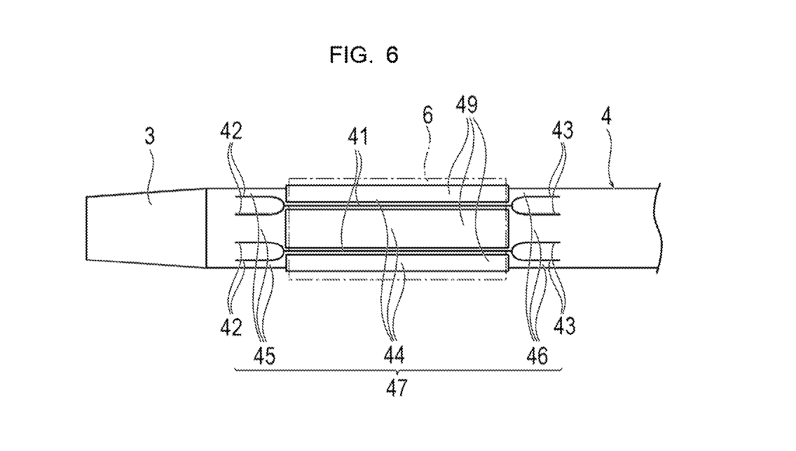

Note that, the configuration of each of the distal side fragile portion 45 and the proximal side fragile portion 46 is not limited as long as flexural rigidity thereof is lower than that of the expansion portion 44. Therefore, for example, as in the modification example illustrated in FIG. 5, the distal side fragile portion 45 and the proximal side fragile portion 46 can have flexural rigidity lower than that of an expansion portion 48 by causing the distal side fragile portion 45, the expansion portion 48, and the proximal side fragile portion 46 to be formed of the same material and causing the thickness of only the expansion portion 48 to be greater than those of the distal side fragile portion 45 and the proximal side fragile portion 46. In this case, the main slit 41, the distal side bifurcated slit 42, and the proximal side bifurcated slit 43 may be formed in a manner similar to the form illustrated in FIG. 2. However, since the flexural rigidity can be set by changing only the thickness, without providing the distal side bifurcated slit 42 and the proximal side bifurcated slit 43 which are bifurcated, the distal side fragile portion 45, the expansion portion 48, and the proximal side fragile portion 46 may be formed so as to have a uniform width in the circumferential direction. In addition, as the material of the expansion portion 44, a material having rigidity higher than that of the material of the distal side fragile portion 45 and the proximal side fragile portion 46 can be adopted. Accordingly, the distal side fragile portion 45 and the proximal side fragile portion 46 can have flexural rigidity lower than that of the expansion portion 44. In addition, as in another modification example illustrated in FIG. 6, the distal side fragile portion 45 and the proximal side fragile portion 46 can be set to have flexural rigidity lower than that of the expansion portion 44 by fixedly attaching a reinforcement member 49 to the expansion portion 44 on the outer circumferential side. Note that, the reinforcement member may be embedded in the expansion portion 44 or may be attached to the expansion portion 44 on the inner circumferential side. The reinforcement member 49 may be formed of the above-described X-ray contrast material.

In this manner, the distal side fragile portion 45 and the proximal side fragile portion 46 can be set to have flexural rigidity lower than that of the expansion portion 44 by causing at least one of the material, the width in the circumferential direction, and the thickness of the distal side fragile portion 45 and the proximal side fragile portion 46 to be different from that of the expansion portion 44.

As illustrated in FIG. 1, the operation unit 5 is provided with an operation unit main body 51 with which the proximal portion of the outer tube 4 is interlocked, and an operation handle 53 which is turnably interlocked with the operation unit main body 51 via a locking screw 52. In the operation unit main body 51, on a side opposite to the portion with which the proximal portion of the outer tube 4 is interlocked, a first finger-hooking portion 54 into which a technician can hook a finger is formed while having the locking screw 52 interposed therebetween. A through-hole is formed in the portion with which the outer tube 4 of the operation unit main body 51 is interlocked, and the inner tube 2 introduced from a proximal side opening portion of the outer tube 4 protrudes toward the proximal side through the through-hole. The operation handle 53 is interlocked with the inner tube 2 protruding toward the proximal side through the through-hole of the operation unit main body 51. The operation handle 53 is bent in a direction away from the operation unit main body 51, at a portion to which the locking screw 52 is connected. On a side opposite to the portion with which the proximal portion of the inner tube 2 is interlocked, a second finger-hooking portion 55 into which a technician can hook a finger is formed while having the locking screw 52 interposed therebetween. When a technician hooks fingers into the first finger-hooking portion 54 and the second finger-hooking portion 55 and causes the first finger-hooking portion 54 and the second finger-hooking portion 55 to be close to each other and to be separated away from each other, the outer tube 4 interlocked with the operation unit main body 51 and the inner tube 2 interlocked with the operation handle 53 can relatively move.

Subsequently, a method of using the expansion device 1 according to the first embodiment will be described by exemplifying a case where the stent 6 is caused to indwell inside a blood vessel.

First, a catheter introducer percutaneously punctures a blood vessel through the Seldinger's method and the like. Subsequently, a guiding catheter in which a guide wire is inserted into a lumen is inserted into the catheter introducer, and the guide wire is preceded. Then, the distal end of the guiding catheter is inserted into a blood vessel through a distal opening of the catheter introducer. Thereafter, while the guide wire is preceded, the guiding catheter is gradually thrust to the target portion.

Subsequently, the tip of the guide wire is inserted into a distal end opening portion of the guide wire lumen 21 of the expansion device 1 in which the contracted stent 6 is installed on the outer circumferential surface of the expansion portion 44 (refer to FIGS. 1 to 3), and the guide wire is drawn out from a proximal end opening portion of the inner tube 2. Subsequently, the expansion device 1 is inserted into the guiding catheter which is inserted into a blood vessel, through the distal member 3. The expansion device 1 is thrust along the guide wire and is caused to protrude from the guiding catheter. The expansion portion 44 is positioned within the target stenosed site. In this case, since the expansion portion 44 includes an X-ray contrast material, the position of the expansion portion 44 can be checked, and positioning can be exactly performed.

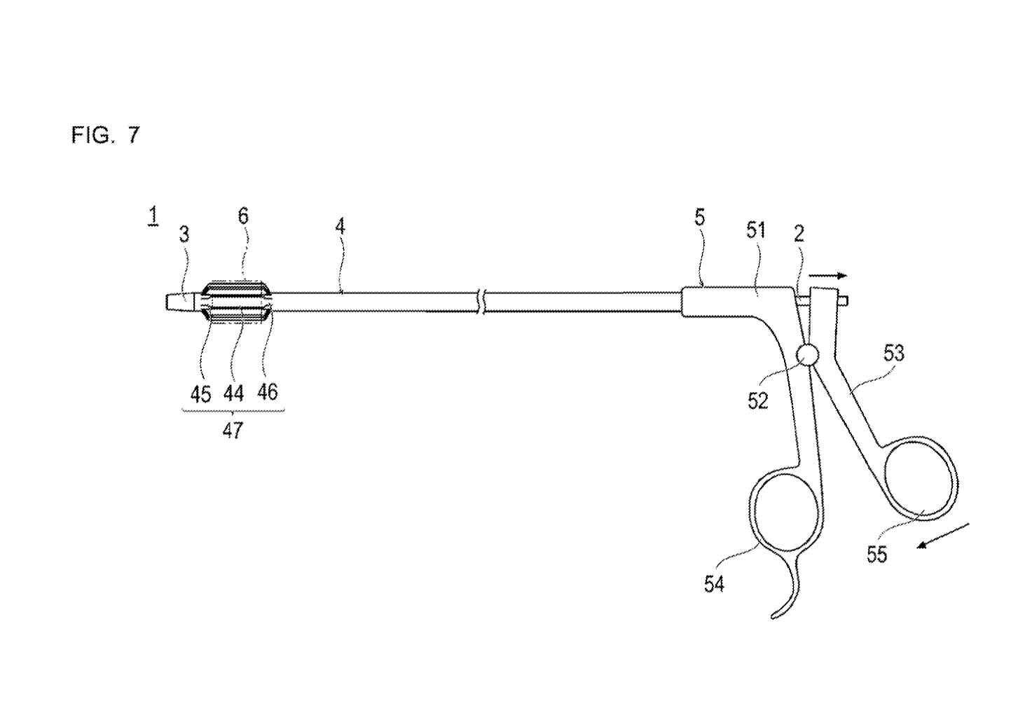

Subsequently, when a technician hooks fingers into the first finger-hooking portion 54 and the second finger-hooking portion 55 and performs an operation of causing the first finger-hooking portion 54 and the second finger-hooking portion 55 to be close to each other, the inner tube 2 moves in the proximal direction with respect to the outer tube 4 as illustrated in FIG. 7. In this case, in order to prevent the position of the stent 6 from being deviated in the axial direction, it can be preferable that the first finger-hooking portion 54 and the second finger-hooking portion 55 are caused to be close to each other by moving both thereof. Since the expansion device 1 according to the present embodiment is provided with the first finger-hooking portion 54 and the second finger-hooking portion 55, movement of both the first finger-hooking portion 54 and the second finger-hooking portion 55 can be subtly adjusted with fingers. Therefore, the position of the stent 6 can be suitably set.

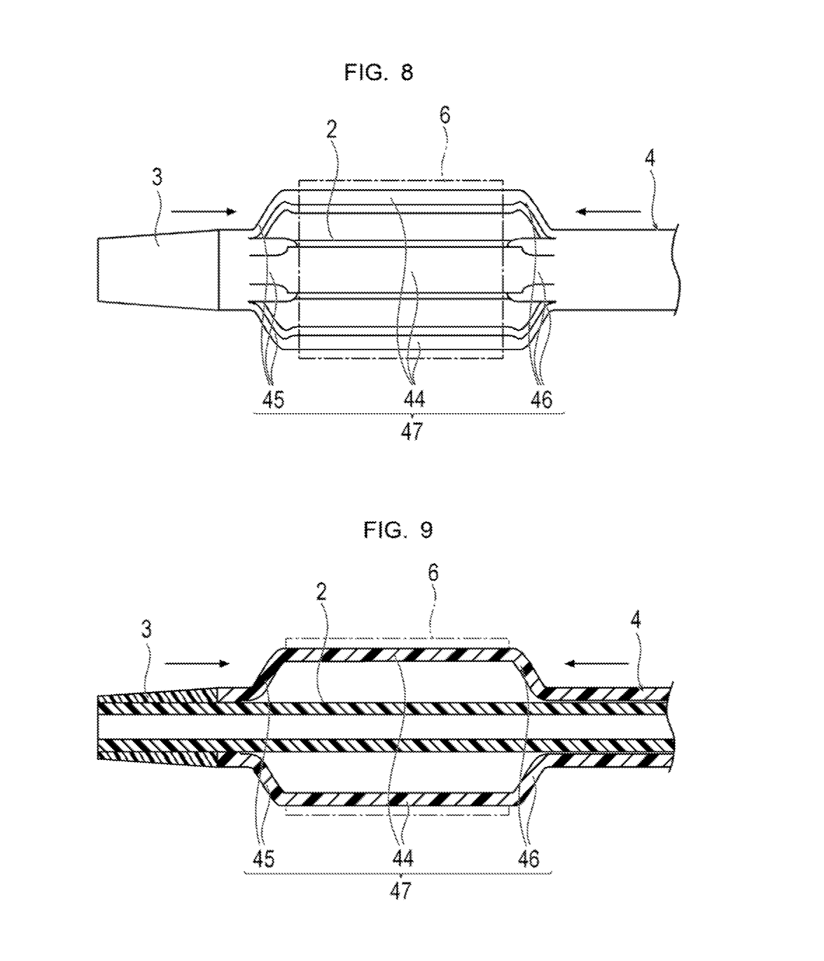

When the inner tube 2 moves in the proximal direction with respect to the outer tube 4, the outer tube 4 is compressed in the axial direction as illustrated in FIGS. 8 and 9. The distal side fragile portion 45 and the proximal side fragile portion 46 warp radially outward, and the expansion portion 44 is widened radially outward. Accordingly, the stent 6 is plastically deformed by the expansion portion 44 and expands, and the stenosed site of a blood vessel is widened. In this case, since the flexural rigidity of the distal side fragile portion 45 and the proximal side fragile portion 46 is lower than the flexural rigidity of the expansion portion 44, the distal side fragile portion 45 and the proximal side fragile portion 46 are mainly curved, and the expansion portion 44 is not curved as much as the distal side fragile portion 45 and the proximal side fragile portion 46. Therefore, in a case where a wall surface of a blood vessel is hardened, the expansion portion 44 of the outer tube 4 is suppressed from being out-pushed by the wall surface and being deformed, and the shape of the expansion portion 44 can be easily and uniformly maintained.

In addition, since the expansion portion 44 expands in a substantially uniform manner and the phenomenon in which only the central portion of the expansion portion 44 significantly protrudes can be suppressed, no excessive load is applied to a vascular wall surface and a load applied to a living body can be reduced. Since the expansion portion 44 is formed in the axial direction and expands in a substantially uniform manner over a relatively wide range, the stent 6 can also be uniformly widened over a wide range.

In addition, when the expansion portion 44 expands, a gap is formed between the wire portions 47 configuring the expansion portions 44. Therefore, the expansion portion 44 does not hinder the blood flow, and a burden to a living body can be reduced. In addition, in a balloon catheter, in order to cause a balloon to expand and contract, a fluid needs to be supplied and discharged. However, since the expansion device 1 according to the present embodiment requires no fluid, a prompt operation can be performed and an influence to a living body can be reduced. In addition, in a case where a balloon is adopted, faulty deflation is likely to occur after the balloon expands such that the fluid cannot be discharged from the balloon. However, since the expansion device 1 according to the present embodiment requires no fluid, the expansion portion 44 can reliably contract.

Thereafter, when a technician performs an operation so as to cause the first finger-hooking portion 54 and the second finger-hooking portion 55 to be separated from each other, the inner tube 2 moves in the distal direction with respect to the outer tube 4. When the inner tube 2 moves in the distal direction with respect to the outer tube 4, compressive force acting on the outer tube 4 decreases, and the expansion portion 44 contracts. Accordingly, the stent 6 can remain in a state of indwelling at the stenosed site of a blood vessel.

Note that, in a case where expansion of the stent 6 is insufficient, the stent 6 can be deformed so as to be in a more desirably state by repeating the above-described operation of the operation unit 5 such that the expansion portion 44 is repeatedly caused to expand. Since the expansion device 1 uses no fluid which is used in a balloon, expansion and contraction of the expansion portion 44 can be easily and promptly repeated.

After the stent 6 indwells inside a blood vessel, the expansion device 1 and the guide wire are evulsed via the guiding catheter. After the guiding catheter is evulsed from the catheter introducer, the catheter introducer is evulsed from a living body, and the technique is completed.

As described above, the expansion device 1 according to the first embodiment can include the outer tube 4 (pipe body) in which a plurality of wire portions 47 extending along the axial direction are disposed so as to be arranged in the circumferential direction in the vicinity of the distal portion, and the inner tube 2 (core) that is inserted into the outer tube 4 and is interlocked with the distal portion of the outer tube 4. The wire portion 47 has the expansion portion 44 which expands radially outward when the outer tube 4 is compressed in the axial direction, the distal side fragile portion 45 which is formed on the distal side of the expansion portion 44 and has flexural rigidity lower than that of the expansion portion 44, and the proximal side fragile portion 46 which is formed on the proximal side of the expansion portion 44 and has flexural rigidity lower than that of the expansion portion 44. Therefore, in a case where compressive force acts on the outer tube 4, the distal side fragile portion 45 and the proximal side fragile portion 46 warp more significantly than the expansion portion 44, and the expansion portion 44 expands radially outward in a substantially uniform manner. Therefore, a load applied to a living body by the expansion portion 44 can be reduced, and a wide range of the expansion portion 44 can uniformly expand.

In addition, the distal side fragile portion 45 and the proximal side fragile portion 46 can be set to have flexural rigidity lower than that of the expansion portion 44 due to at least one of the material, the width in the circumferential direction, and the thickness different from that of the expansion portion 44. Therefore, the flexural rigidity of the distal side fragile portion 45 and the proximal side fragile portion 46 can be set easily and suitably.

In addition, in the outer tube 4 (pipe body), the main slit 41 is formed between the expansion portions 44 adjacent to each other in the circumferential direction, a plurality of distal side bifurcated slits 42 and a plurality of proximal side bifurcated slits 43 (bifurcated slits) bifurcated respectively from the distal side and the proximal side of the main slit 41 are formed, and the widths of the distal side fragile portion 45 and the proximal side fragile portion 46 in the circumferential direction are set to be smaller than that of the expansion portion 44 due to the distal side bifurcated slits 42 and the proximal side bifurcated slits 43. Therefore, the flexural rigidity of the expansion portion 44, the distal side fragile portion 45, and the proximal side fragile portion 46 can be set easily and suitably by only forming the main slit 41, the distal side bifurcated slits 42, and the proximal side bifurcated slits 43.

In addition, when the expansion portion 44 includes an X-ray contrast material, the position of the expansion portions 44 positioned inside a biological lumen can be checked, and the expansion portions 44 can expand at the exact position.

In addition, when the inner tube 2 (core) includes an X-ray contrast material in a portion positioned on the inner side of the expansion portion 44, the position of the expansion portions 44 positioned inside a biological lumen can be checked, and the expansion portions 44 can expand at the exact position.

In an expansion device 7 according to a second embodiment of the present disclosure, only the configuration of an operation unit 71 is different from that of the first embodiment. Note that, the same reference sign will be applied to a portion having the same function as that of the first embodiment, and the description thereof will be omitted.

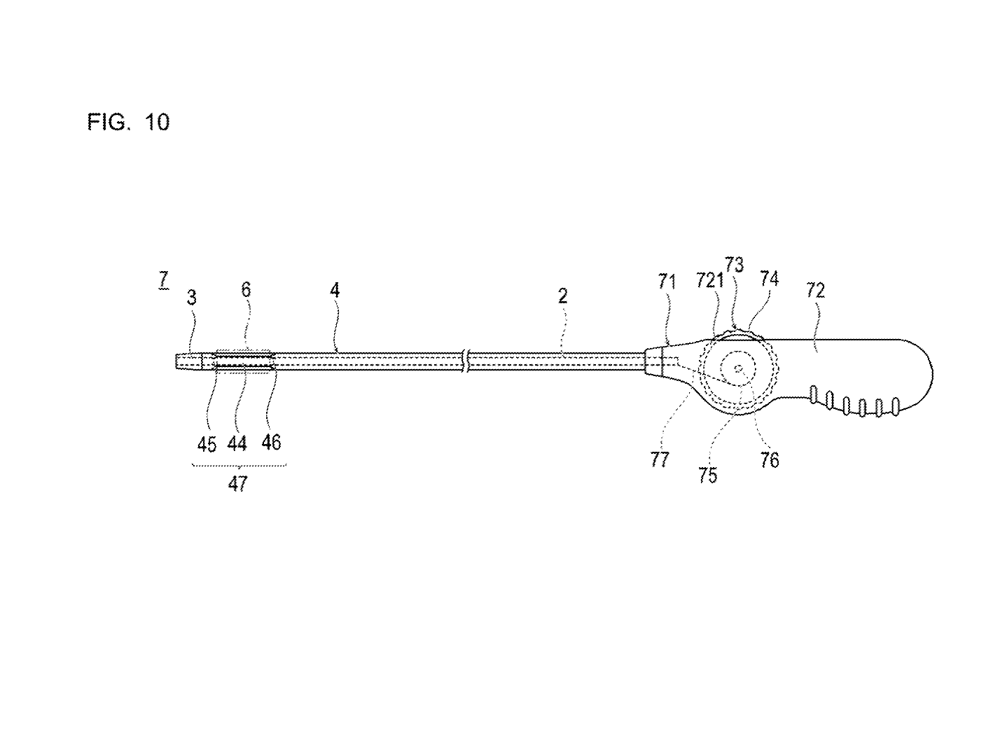

As illustrated in FIG. 10, the expansion device 7 according to the second embodiment is provided with a housing 72 to which the proximal portion of the outer tube 4 is fixed, a rotative operation unit 73 which is rotatably disposed inside the housing 72, and an operation wire 77.

The housing 72 is formed so as to have a shape a technician can easily hold, and a part of the rotative operation unit 73 is exposed through an opening portion 721 formed in the housing 72.

The rotative operation unit 73 is provided with a disk-shaped the operation dial 74, a columnar winding portion 75 which is integrally and coaxially formed with an operation dial 74 and can wind the operation wire 77 around the outer circumferential surface, and an axis portion 76 which is positioned on the central axis of the operation dial 74 and is rotatably and pivotally attached to the housing 72. The operation dial 74 is a portion which is rotatively operated by a technician with a finger, and a part of the outer circumference is exposed to the outside through the opening portion 721 of the housing 72 such that the technician can operate.

One end of the operation wire 77 is fixed to the proximal portion of the inner tube 2, and the other end is fixed to the outer circumferential surface of the winding portion 75.

Subsequently, an operation of the expansion device 7 according to the second embodiment will be described.

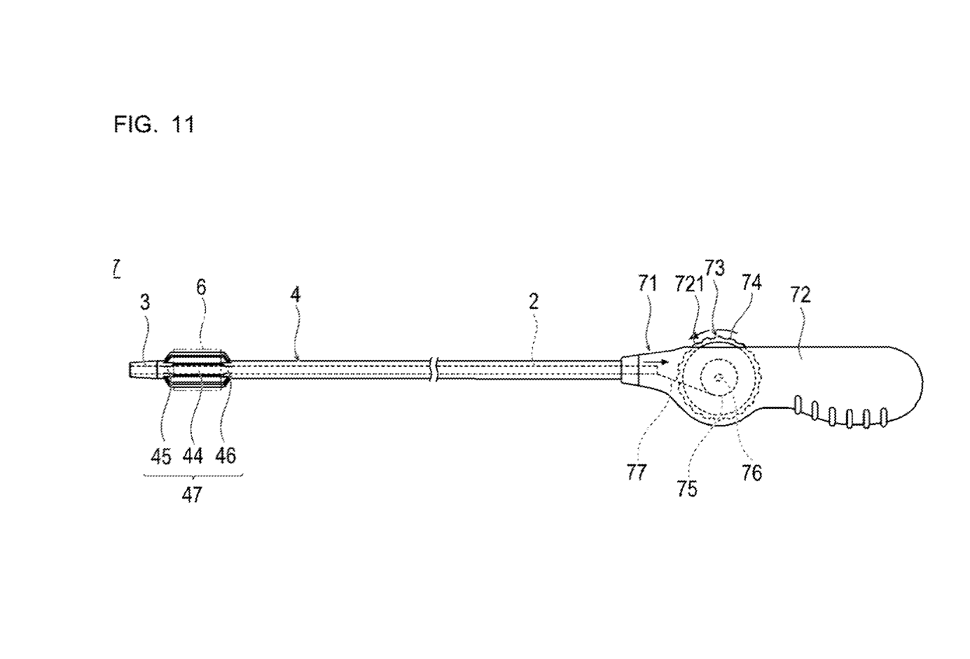

In the expansion device 7 according to the second embodiment, when a technician rotates the operation dial 74, the operation wire 77 is wound around the winding portion 75, and the inner tube 2 fixed to the operation wire 77 moves in the proximal direction with respect to the outer tube 4. When the inner tube 2 moves in the proximal direction with respect to the outer tube 4, the outer tube 4 is compressed in the axial direction as illustrated in FIG. 11. The distal side fragile portion 45 and the proximal side fragile portion 46 warp radially outward, and the expansion portion 44 is widened radially outward. Accordingly, the stent 6 is plastically deformed by the expansion portion 44 and expands, and for example, the stenosed site of a blood vessel is widened.

Thereafter, when the technician rotates the operation dial 74 in the opposite direction, the operation wire 77 wound around the winding portion 75 moves in the distal direction, the inner tube 2 fixed to the operation wire 77 moves in the distal direction with respect to the outer tube 4. When the inner tube 2 moves in the distal direction with respect to the outer tube 4, compressive force acting on the outer tube 4 decreases, and the expansion portion 44 contracts. Accordingly, the stent 6 can remain in a state of indwelling at the stenosed site of a blood vessel.

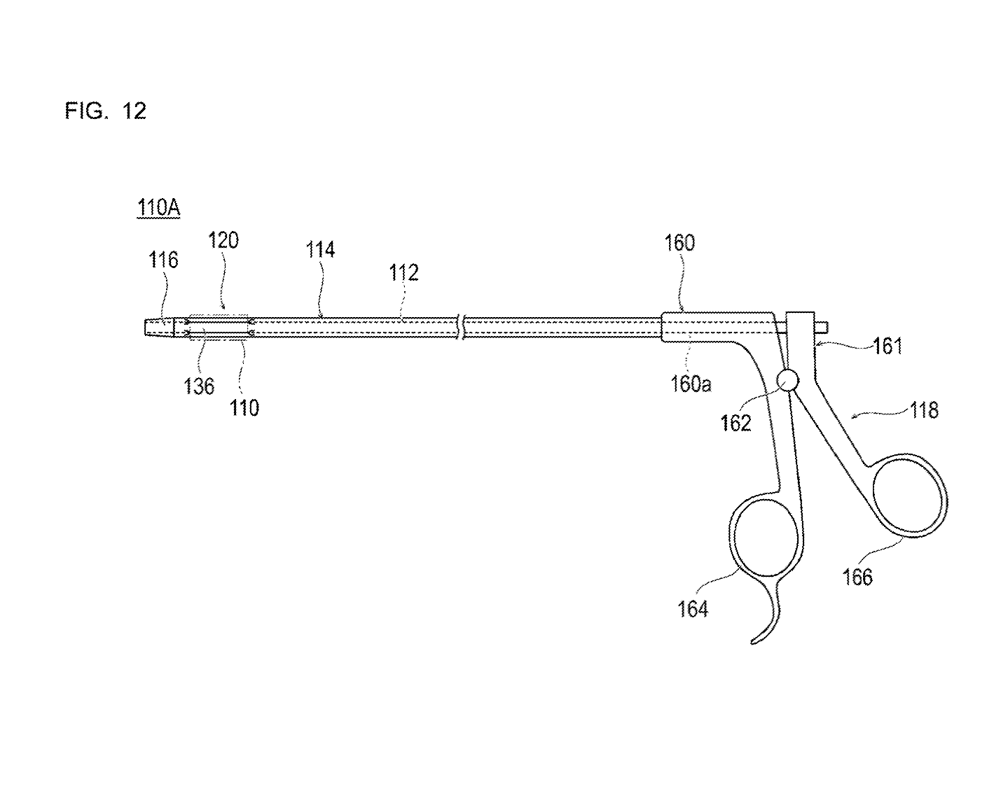

In an expansion device 110A according to a third embodiment of the present disclosure, as illustrated in FIGS. 12 to 15, the structure of an expansion mechanism 120 of the distal portion is different from that of the first embodiment.

For example, the expansion device 110A according to the third embodiment is used in order to improve a lesion area by delivering a stent 111 to the lesion area such as a stenosed site and an occluded site generated inside a blood vessel, the bile duct, the trachea, the esophagus, the urethra, or other biological lumens, causing the stent 111 to expand at the place thereof, and widening the lesion area. Otherwise, the expansion device 110A can also be used in order to improve a lesion area by directly widening the lesion area generated inside a biological lumen. In addition, the expansion device 110A can also be utilized as a backup auxiliary anchor when a device such as a guide wire passes through a lesion area such as stenosed site and an occluded site.

The expansion device 110A is provided with a tubular inner tube 112 (core), a tubular outer tube 114 (pipe body) which has the expansion mechanism 120 able to expand and contract and covers the inner tube 112, a distal member 116 which is fixedly attached to the outer circumferential surface of the distal portion of the inner tube 112, and an operation unit 118 to which the proximal portions of the inner tube 112 and the outer tube 114 are connected.

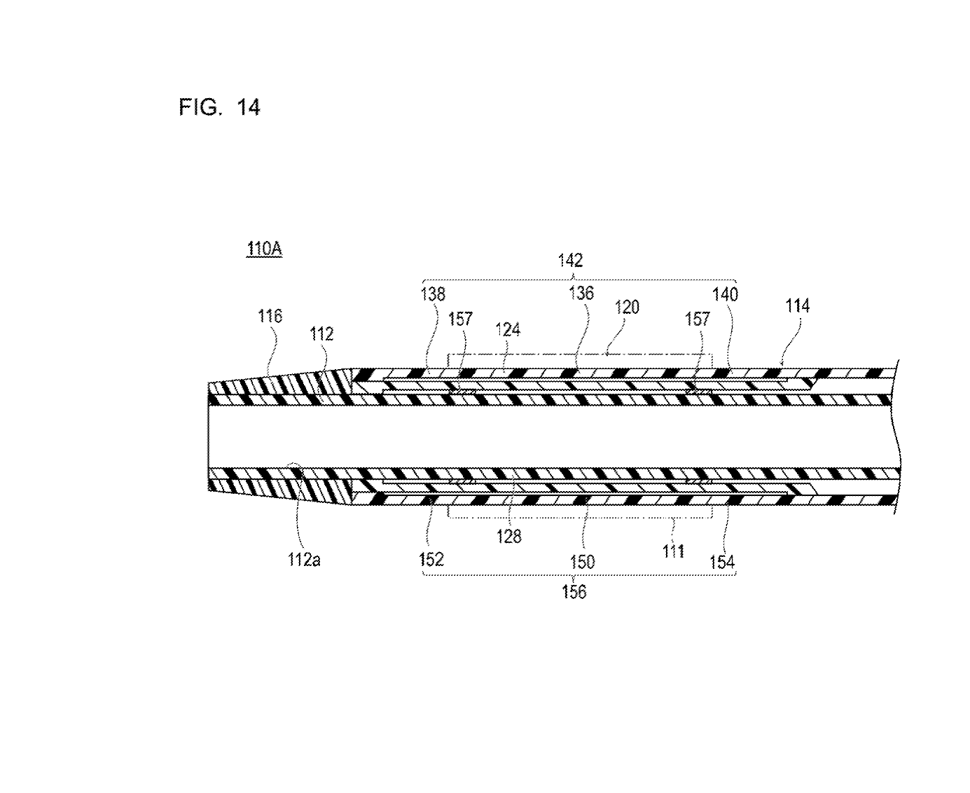

As illustrated in FIG. 14, the inner tube 112 is disposed inside the outer tube 114 and is interlocked with the outer tube 114 on the distal side closer than the expansion mechanism 120 of the outer tube 114. The guide wire is inserted into a guide wire lumen 112a formed in the inner tube 112.

The outer tube 114 can relatively move in the axial direction with respect to the inner tube 112. The distal portion of the outer tube 114 can be fixedly attached to the outer circumferential surface in the vicinity of the distal portion of the inner tube 112. Note that, the distal portion of the outer tube 114 may fixedly attached to the inner tube 112 via the distal member 116. In addition, the distal member 116 may be a part of the inner tube 112, or may be a part of the outer tube 114.

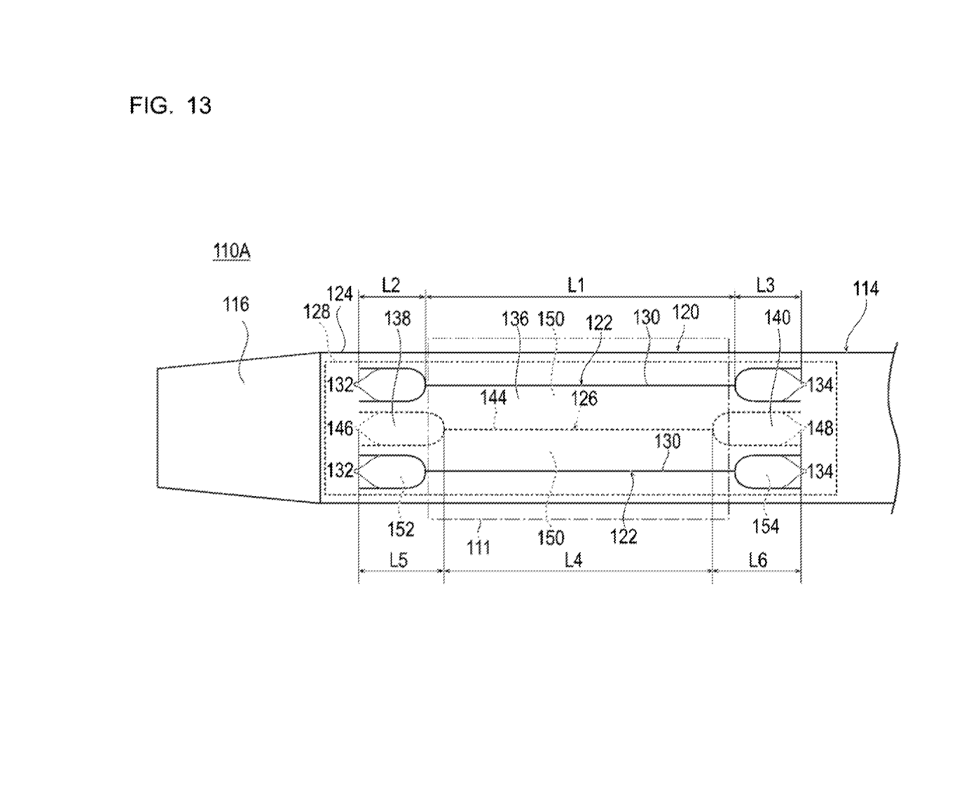

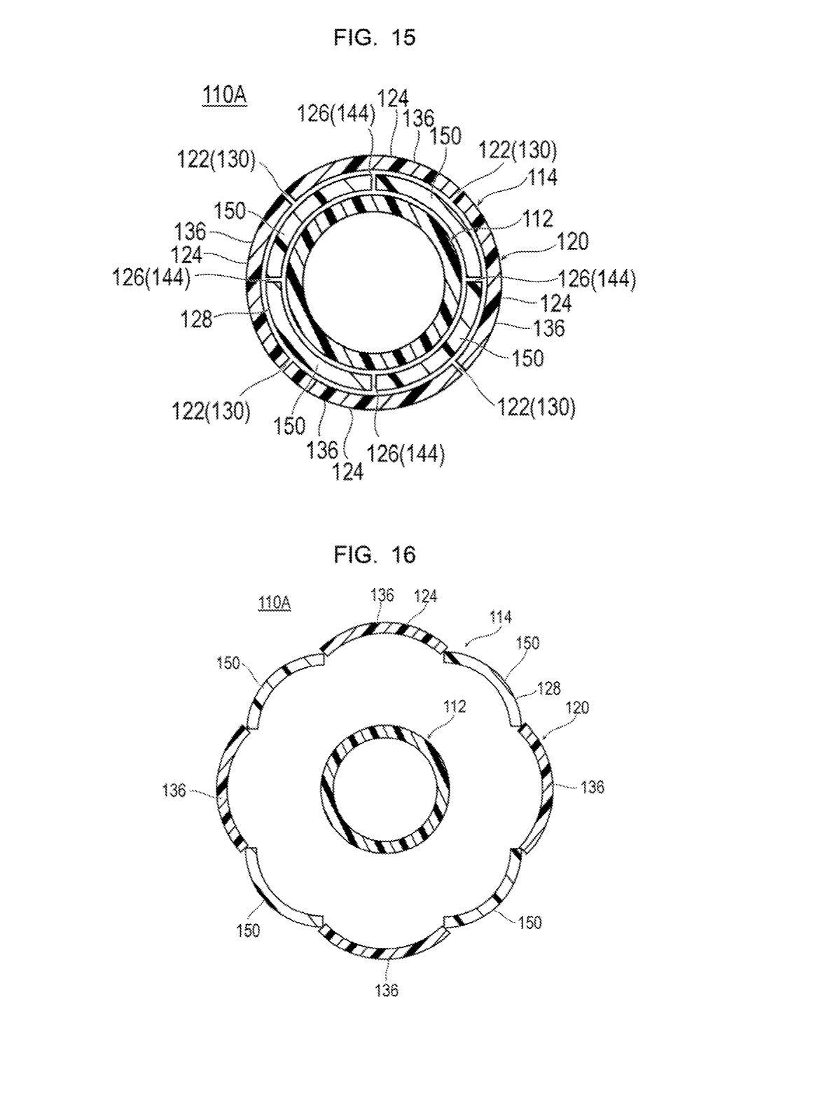

FIG. 15 is a cross-sectional view of the expansion mechanism 120 in a contraction state, and FIG. 16 is a cross-sectional view of the expansion mechanism 120 in an expansion state. In FIGS. 13 to 16, the outer tube 114 has a first pipe portion 124 in which a plurality of (four in the illustrated example) first slits 122 are formed with space therebetween in the circumferential direction, and a second pipe portion 128 which is disposed between the first pipe portion 124 and the inner tube 112 and in which a plurality of (four in the illustrated example) second slits 126 are formed with space therebetween in the circumferential direction.

The first pipe portion 124 has the same length as the overall length of the outer tube 114 and extends from the proximal end of the distal member 116 to the operation unit 118. The second pipe portion 128 has a length shorter than that of the first pipe portion 124 and is disposed inside the distal portion of the first pipe portion 124. The distal portion of the second pipe portion 128 is bonded to the outer circumferential surface in the vicinity of the distal portion of the inner tube 112, the proximal surface of the distal member 116, and the inner circumferential surface of the distal portion of the first pipe portion 124. The proximal portion of the second pipe portion 128 is bonded to the inner circumferential surface of the first pipe portion 124.

Note that, in place of the configuration in the illustrated example, the second pipe portion 128 may have the same length as the overall length of the outer tube 114 and may extend from the proximal end of the distal member 116 to the operation unit 118, and the first pipe portion 124 may have a length shorter than that of the second pipe portion 128 and may be disposed outside the distal portion of the second pipe portion 128.

As illustrated in FIG. 13, each of the first slits 122 has a main slit 130 extending along the axial direction, a plurality of (two in the present embodiment) distal side bifurcated slits 132 (bifurcated slits) continuously widened and bifurcated from the main slit 130 toward the distal direction, and a plurality of (two in the present embodiment) proximal side bifurcated slits 134 (bifurcated slits) continuously widened and bifurcated from the main slit 130 toward the proximal direction. In the present embodiment, the main slit 130 is formed so as to have equal spaces among them in the circumferential direction and to be parallel to the axial line of the outer tube 114.

In the first pipe portion 124, between the main slits 130 adjacent to each other in the circumferential direction, a first expansion portion 136 (expansion portion) having a width in the circumferential direction uniformly formed in the axial direction is formed. The first expansion portion 136 is a portion where the stent 111 is placed.

On the distal side of the first expansion portion 136, a first distal side fragile portion 138 (distal side fragile portion) which continues from the first expansion portion 136 is formed so as to be interposed between the distal side bifurcated slits 132 adjacent to each other in the circumferential direction. Since the first distal side fragile portion 138 is formed so as to be interposed between the distal side bifurcated slits 132 widened and bifurcated from the main slit 130, the width in the circumferential direction is formed so as to be smaller than that of the first expansion portion 136.

On: the proximal side of the first expansion portion 136, a first proximal side fragile portion 140 (proximal side fragile portion) which continues from the first expansion portion 136 is formed so as to be interposed between the proximal side bifurcated slits 134 adjacent to each other in the circumferential direction. Since the first proximal side fragile portion 140 is formed so as to be interposed between the proximal side bifurcated slits 134 widened and bifurcated from the main slit 130, the width in the circumferential direction is formed so as to be smaller than that of the first expansion portion 136.

As illustrated in FIG. 14, the first distal side fragile portion 138, the first expansion portion 136, and the first proximal side fragile portion 140 form a first wire portion 142 (wire portion) continuously extending along the axial line of the outer tube 114. Therefore, in the first pipe portion 124, a plurality of the first wire portions 142 are formed so as to be arranged in the circumferential direction.

The first distal side fragile portion 138, the first expansion portion 136, and the first proximal side fragile portion 140 are formed so as to have a uniform thickness by using the same material. Therefore, the first distal side fragile portion 138 and the first proximal side fragile portion 140 having a width in the circumferential direction smaller than that of the first expansion portion 136 have flexural rigidity lower than that of the first expansion portion 136.

As illustrated in FIG. 13, similar to the first slit 122, each of the second slits 126 has a main slit 144, a plurality of (two in the present embodiment) distal side bifurcated slits 146 (bifurcated slits), and a plurality of (two in the present embodiment) proximal side bifurcated slits 148 (bifurcated slits). In the present embodiment, the main slit 144 is formed so as to have equal spaces among them in the circumferential direction and to be parallel to the axial line of the outer tube 114.

In addition, similar to the first pipe portion 124, in the second pipe portion 128, a second expansion portion 150 (expansion portion) having a uniformly formed width in the circumferential direction is formed between the main slits 144 adjacent to each other in the circumferential direction, a second distal side fragile portion 152 (distal side fragile portion) having a width in the circumferential direction smaller than that of the second expansion portion 150 is formed on the distal side of the second expansion portion 150, and a second proximal side fragile portion 154 (proximal side fragile portion) having a width in the circumferential direction smaller than that of the second expansion portion 150 is formed on the proximal side of the second expansion portion 150.

The second distal side fragile portion 152, the second expansion portion 150, and the second proximal side fragile portion 154 are formed so as to have a uniform thickness by using the same material. Therefore, the second distal side fragile portion 152 and the second proximal side fragile portion 154 having a width in the circumferential direction smaller than that of the second expansion portion 150 have flexural rigidity lower than that of the first expansion portion 136.

As illustrated in FIG. 14, the second distal side fragile portion 152, the second expansion portion 150, and the second proximal side fragile portion 154 form a second wire portion 156 (wire portion) continuously extending along the axial line. Therefore, in the second pipe portion 128, a plurality of second wire portions 156 are formed so as to be arranged in the circumferential direction.

As illustrated in FIG. 15, the plurality of first slits 122 (particularly, the main slit 130) and the plurality of second slits 126 (particularly, the main slit 144) are formed at positions different from each other in the circumferential direction. In the present embodiment, at the center angle position of the main slit 130 between the first slits 122 adjacent to each other in the circumferential direction, the main slit 144 of the second slits 126 is formed. Accordingly, the main slit 130 and the main slit 144 are deviated from each other in the circumferential direction by 45° (45 degrees).

Note that, the slit denotes a cut-off portion or an opening portion which extends linearly. Therefore, the first slits 122 and the second slits 126 may be opening portions through each of which a member inside thereof is exposed. The number of the first slits 122 and the number of the second slits 126 are not limited as long as there are two or more for each of the first slits 122 and the second slits 126. The first slits 122 and the second slits 126 are not necessarily formed so as to have equal spaces among them in the circumferential direction. The main slits 130 and 144 may be formed so as to incline with respect to the axial line of the outer tube 114.

The expansion mechanism 120 provided in the outer tube 114 is configured to have the plurality of first wire portions 142 formed in the first pipe portion 124 and the plurality of second wire portions 156 formed in the second pipe portion 128 which are described above.

As illustrated in FIG. 13, in the present embodiment, a length L5 of the second distal side fragile portion 152 is slightly longer than a length L2 of the first distal side fragile portion 138 as much as (ΔL), and a length L6 of the second proximal side fragile portion 154 is slightly longer than a length L3 of the first proximal side fragile portion 140 as much as (ΔL). Therefore, a length L4 of the second expansion portion 150 is shorter than a length L1 of the first expansion portion 136 as much as 2ΔL. In this case, in an expansion state of the expansion mechanism 120 (FIG. 16), ΔL is set such that the first expansion portions 136 and the second expansion portions 150 are alternately arranged in the circumferential direction.

As the material forming the inner tube 112 and the outer tube 114, a material similar to those of the inner tube 2 and the outer tube 4 in the first embodiment can be used.

Note that, the configuration of each of the first distal side fragile portion 138 and the first proximal side fragile portion 140 is not limited as long as flexural rigidity thereof is lower than that of the first expansion portion 136. Similarly, the configuration of each of the second distal side fragile portion 152 and the second proximal side fragile portion 154 is not limited as long as flexural rigidity thereof is lower than that of the second expansion portion 150.

Therefore, for example, the first distal side fragile portion 138 and the first proximal side fragile portion 140 can have flexural rigidity lower than that of the first expansion portion 136 by causing the first distal side fragile portion 138, the first expansion portion 136, and the first proximal side fragile portion 140 to be formed of the same material and causing the thickness of only the first expansion portion 136 to be greater than those of the first distal side fragile portion 138 and the first proximal side fragile portion 140. In this case, the main slit 130, the distal side bifurcated slit 132, and the proximal side bifurcated slit 134 may be formed in a manner similar to the form illustrated in FIG. 13. However, since the flexural rigidity can be set by changing only the thickness, without providing the distal side bifurcated slit 132 and the proximal side bifurcated slit 134 which are bifurcated, the first distal side fragile portion 138, the first expansion portion 136, and the first proximal side fragile portion 140 may be formed so as to have a uniform width in the circumferential direction.

Otherwise, as the material of the first expansion portion 136, a material having rigidity higher than that of the material of the first distal side fragile portion 138 and the first proximal side fragile portion 140 can be adopted. Accordingly, the first distal side fragile portion 138 and the first proximal side fragile portion 140 can have flexural rigidity lower than that of the first expansion portion 136. Otherwise, the first distal side fragile portion 138 and the first proximal side fragile portion 140 can be set to have flexural rigidity lower than that of the first expansion portion 136 by fixedly attaching a reinforcement member to the first expansion portion 136 on the outer circumferential side. Note that, the reinforcement member may be embedded in the first expansion portion 136 or may be attached to the first expansion portion 136 on the inner circumferential side. The reinforcement member may be formed of an X-ray contrast material.

In this manner, the first distal side fragile portion 138 and the first proximal side fragile portion 140 can be set to have flexural rigidity lower than that of the first expansion portion 136 by causing at least one of the material, the width in the circumferential direction, and the thickness of the first distal side fragile portion 138 and the first proximal side fragile portion 140 to be different from that of the first expansion portion 136. Similarly, the second distal side fragile portion 152 and the second proximal side fragile portion 154 can be set to have flexural rigidity lower than that of the second expansion portion 150 by causing at least one of the material, the width in the circumferential direction, and the thickness of the second distal side fragile portion 152 and the second proximal side fragile portion 154 to be different from that of the second expansion portion 150.

In FIGS. 12 to 14, the distal member 116 is a member configuring the outermost distal portion of the expansion device 110A and is fixedly attached to the outer circumferential surface of the distal portion of the inner tube 112. In order to reduce an influence to a living body when the expansion device 110A is inserted into a lumen, the distal member 116 is formed of a flexible material and is decreased in diameter toward the distal direction.

As the material forming the distal member 116, a material similar to that of the distal member 3 in the first embodiment can be used.

The length of the distal member 116 from the outermost distal portion to the operation unit 118 is not particularly limited. For example, in a case where the distal member 116 is adopted in expansion of the blood vessel stent 111, the length, for example, preferably ranges from 1,200 to 1,600 mm. The outer diameter of the outer tube 114 is not particularly limited. For example, in a case where the outer tube 114 is adopted in expansion of the blood vessel stent 111, the thickness, for example, preferably ranges from 0.5 to 2.0 mm.

The expansion mechanism 120 may be formed so as to include an X-ray contrast material in the configuration material. Accordingly, the position of the stent 111 can be accurately grasped under X-ray contrast condition, and thus, the technique is more easily performed. As the X-ray contrast material, for example, gold, platinum, a platinum-iridium alloy, silver, stainless steel, molybdenum, tungsten, tantalum, palladium, or an alloy thereof is favorable.

As in FIG. 14, a contrast marker 157 formed of an X-ray contrast material may be disposed at a position surrounded by the first expansion portions 136 in the inner tube 112. The contrast marker 157 is attached by winding a wire formed of an X-ray contrast material around the outer surface of the inner tube 112 or caulking or gluing a pipe formed of an X-ray contrast material onto the outer surface of the inner tube 112.

As illustrated in FIG. 12, the operation unit 118 is provided with an operation unit main body 160 with which the proximal portion of the outer tube 114 is interlocked, and an operation handle 161 which is turnably interlocked with the operation unit main body 160 via a locking screw 162. In the operation unit main body 160, on a side opposite to the portion with which the proximal portion of the outer tube 114 is interlocked, a first finger-hooking portion 164 into which a technician can hook a finger is formed while having the locking screw 162 interposed there between.

A through-hole 160a is formed in the portion with which the outer tube 114 of the operation unit main body 160 is interlocked, and the inner tube 112 introduced from the proximal side opening portion of the outer tube 114 protrudes toward the proximal side through the through-hole 160a. The operation handle 161 is interlocked with the inner tube 112 protruding toward the proximal side through the through-hole 160a of the operation unit main body 160. The operation handle 161 is bent in a direction away from the operation unit main body 160, at a portion to which the locking screw 162 is connected. On a side opposite to the portion with which the proximal portion of the inner tube 112 is interlocked, a second finger-hooking portion 166 into which a technician can hook a finger is formed while having the locking screw 162 interposed therebetween.

When a technician hooks fingers into the first finger-hooking portion 164 and the second finger-hooking portion 166 and causes the first finger-hooking portion 164 and the second finger-hooking portion 166 to be close to each other and to be separated away from each other, the outer tube 114 interlocked with the operation unit main body 160 and the inner tube 112 interlocked with the operation handle 161 can relatively move.

The medical device according to the present embodiment basically has the configured as described above. Hereinafter, an operation and an effect thereof will be described.

In treatment in which the expansion device 110A is adopted, in a case of treating a lesion area (stenosed site) generated inside a blood vessel, for example, the treatment is performed as follows. First, the form of the lesion area (stenosed site) generated inside a blood vessel is specified through an intravascular contrast method or an intravascular ultrasound diagnostic method.

Subsequently, a catheter introducer percutaneously punctures a blood vessel through the Seldinger's method and the like. Subsequently, a guiding catheter in which a guide wire is inserted into a lumen is inserted into the catheter introducer, and the guide wire is preceded. Then, the distal end of the guiding catheter is inserted into a blood vessel through a distal opening of the catheter introducer. Under radioscopic condition, while the guide wire is preceded, the guiding catheter is gradually thrust to the target portion.

Subsequently, the tip of the guide wire is inserted into a distal end opening portion of the guide wire lumen 112a of the expansion device 110A in which the contracted stent 111 is installed on the outer circumferential surface of the expansion mechanism 120 (specifically, the first expansion portion 136), and the guide wire is drawn out from a proximal end opening portion of the inner tube 112. Subsequently, the expansion device 110A is inserted into the guiding catheter which is inserted into a blood vessel, through the distal member 116. The expansion device 110A is thrust along the guide wire and is caused to protrude from the guiding catheter. The first expansion portion 136 is positioned within the lesion area. In this case, since the expansion mechanism 120 includes an X-ray contrast material, the position of the first expansion portion 136 can be checked, and positioning can be exactly performed.

Subsequently, when a technician hooks fingers into the first finger-hooking portion 164 and the second finger-hooking portion 166 and performs an operation of causing the first finger-hooking portion 164 and the second finger-hooking portion 166 to be close to each other, the inner tube 112 moves in the proximal direction with respect to the outer tube 114. In this case, in order to prevent the position of the stent 111 from being deviated in the axial direction, it is preferable that the first finger-hooking portion 164 and the second finger-hooking portion 166 are caused to be close to each other by moving both thereof.

Since the expansion device 110A according to the present embodiment is provided with the first finger-hooking portion 164 and the second finger-hooking portion 166, movement of both the first finger-hooking portion 164 and the second finger-hooking portion 166 can be subtly adjusted with fingers. Therefore, the position of the stent 111 can be suitably set.

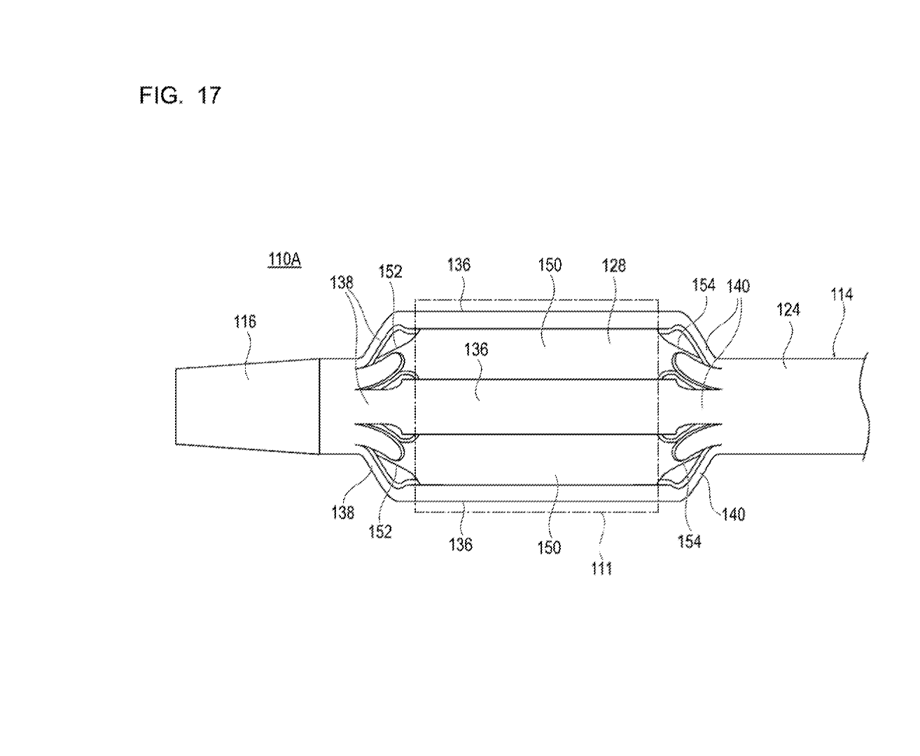

When the inner tube 112 moves in the proximal direction with respect to the outer tube 114, the first pipe portion 124 and the second pipe portion 128 configuring the outer tube 114 are compressed in the axial direction. Accordingly, as illustrated in FIGS. 16 and 17, in the first pipe portion 124, the first distal side fragile portion 138 and the first proximal side fragile portion 140 warp radially outward, and the first expansion portion 136 is widened radially outward. In addition, in the second pipe portion 128, the second distal side fragile portion 152 and the second proximal side fragile portion 154 warp radially outward, and the second expansion portion 150 is widened radially outward.

Accordingly, the stent 111 is plastically deformed by a plurality of the first expansion portions 136 and a plurality of the second expansion portions 150 and expands, and the lesion area (stenosed site) is widened. In this case, in the present embodiment, since the flexural rigidity of the first distal side fragile portion 138 and the first proximal side fragile portion 140 is lower than the flexural rigidity of the first expansion portion 136, the first distal side fragile portion 138 and the first proximal side fragile portion 140 are mainly curved, and the first expansion portion 136 is not curved as much as the first distal side fragile portion 138 and the first proximal side fragile portion 140. Similarly, since the flexural rigidity of the second distal side fragile portion 152 and the second proximal side fragile portion 154 is lower than the flexural rigidity of the second expansion portion 150, the second distal side fragile portion 152 and the second proximal side fragile portion 154 are mainly curved, and the second expansion portion 150 is not curved as much as the second distal side fragile portion 152 and the second proximal side fragile portion 154. Therefore, in a case where a wall surface of a blood vessel is hardened, the first expansion portion 136 and the second expansion portion 150 are suppressed from being out-pushed by the wall surface and being deformed, and the shapes of the first expansion portion 136 and the second expansion portion 150 can be easily and uniformly maintained.

In addition, in the first expansion portion 136 and the second expansion portion 150, the phenomenon in which the central portion in the axial direction significantly protrudes can be suppressed. Therefore, even in a case where an intravascular wall is directly pressed by the first expansion portion 136 and the second expansion portion 150, no excessive load is applied to the intravascular wall and a load applied to a living body can be reduced. Since a wide range in which the first expansion portion 136 and the second expansion portion 150 are formed in the axial direction expands in a substantially uniform manner, a wide range of the stent 111 can be uniformly widened.

In a balloon catheter, in order to cause a balloon to expand and contract, a fluid needs to be supplied and discharged. However, since the expansion device 110A according to the present embodiment requires no fluid, a prompt operation can be performed and an influence to a living body can be reduced.

After the stent 111 is caused to expand as described above, when a technician performs an operation so as to cause the first finger-hooking portion 164 and the second finger-hooking portion 166 to be separated from each other, the inner tube 112 moves in the distal direction with respect to the outer tube 114. When the inner tube 112 moves in the distal direction with respect to the outer tube 114, compressive force acting on the outer tube 114 decreases, and the expansion mechanism 120 contracts. Accordingly, the stent 111 can remain inside a living body in a state of indwelling at the stenosed site of a blood vessel.

Note that, in a case where expansion of the stent 111 is insufficient, the stent 11 can be deformed so as to be in a more desirably state by repeating the above-described operation of the operation unit 118 such that the expansion mechanism 120 is repeatedly caused to expand. Since the expansion device 110A uses no fluid which is used in a balloon, expansion and contraction of the expansion mechanism 120 can be easily and promptly repeated.

After the stent 111 indwells inside a blood vessel, the expansion device 110A and the guide wire are evulsed via the guiding catheter. After the guiding catheter is evulsed from the catheter introducer, the catheter introducer is evulsed from a living body, and the technique is completed.

As described above, according to the expansion device 110A, the expansion mechanism 120 expands in response to compressive force acting in the axial direction of the outer tube 114 due to relative movement between the outer tube 114 and the inner tube 112 in the axial direction. Thus, an expansion target can be caused to expand without adopting a dilation fluid.

In addition, in a contraction state, the expansion mechanism 120 has a dual structure having the first expansion portions 136 on the outer side and the second expansion portions 150 on the inner side, and the plurality of first slits 122 and the plurality of second slits 126 are formed at positions different from each other in the circumferential direction. Therefore, while the expansion mechanism 120 is in an expansion state, due to the first expansion portions 136 and the second expansion portions 150 arranged in the circumferential direction, the expansion target can be pressed from the inside over a wide range in the circumferential direction. Accordingly, the expansion target can be uniformly widened in the circumferential direction.

In addition, while the expansion mechanism 120 is in an expansion state, the first expansion portions 136 and the second expansion portions 150 are alternately arranged in the circumferential direction, the expansion target can be more uniformly widened in the circumferential direction.

In the case of the present embodiment, in response to compressive forces acting in the axial direction of the first pipe portion 124, the first distal side fragile portion 138 and the first proximal side fragile portion 140 warp more significantly than the first expansion portion 136. Thus, the first expansion portion 136 expands radially outward in a substantially uniform manner in the axial direction, in addition, a biological lumen is not completely occluded when the expansion mechanism 120 expands. Thus, the expansion target can be caused to expand without interrupting a flow of a body fluid such as blood. In addition, in response to compressive forces acting in the axial direction of the second pipe portion 128, the second distal side fragile portion 152 and the second proximal side fragile portion 154 warp more significantly than the second expansion portion 150. Thus, the second expansion portion 150 expands radially outward in a substantially uniform manner in the axial direction. Therefore, a wide range of the expansion mechanism 120 in the axial direction can uniformly expand.

Particularly, in the case of the present embodiment, the second distal side fragile portion 152 has a length in the axial direction slightly longer than that of the first distal side fragile portion 138, and the second proximal side fragile portion 154 has a length in the axial direction slightly longer than that of the first proximal side fragile portion 140. According to the configuration, while the expansion mechanism 120 is in an expansion state, the outer diameter formed with the plurality of first expansion portions 136 and the outer diameter formed with the plurality of second expansion portions 150 can be substantially equal to each other. Thus, the expansion mechanism 120 can expand in a substantially uniform manner in the circumferential direction.

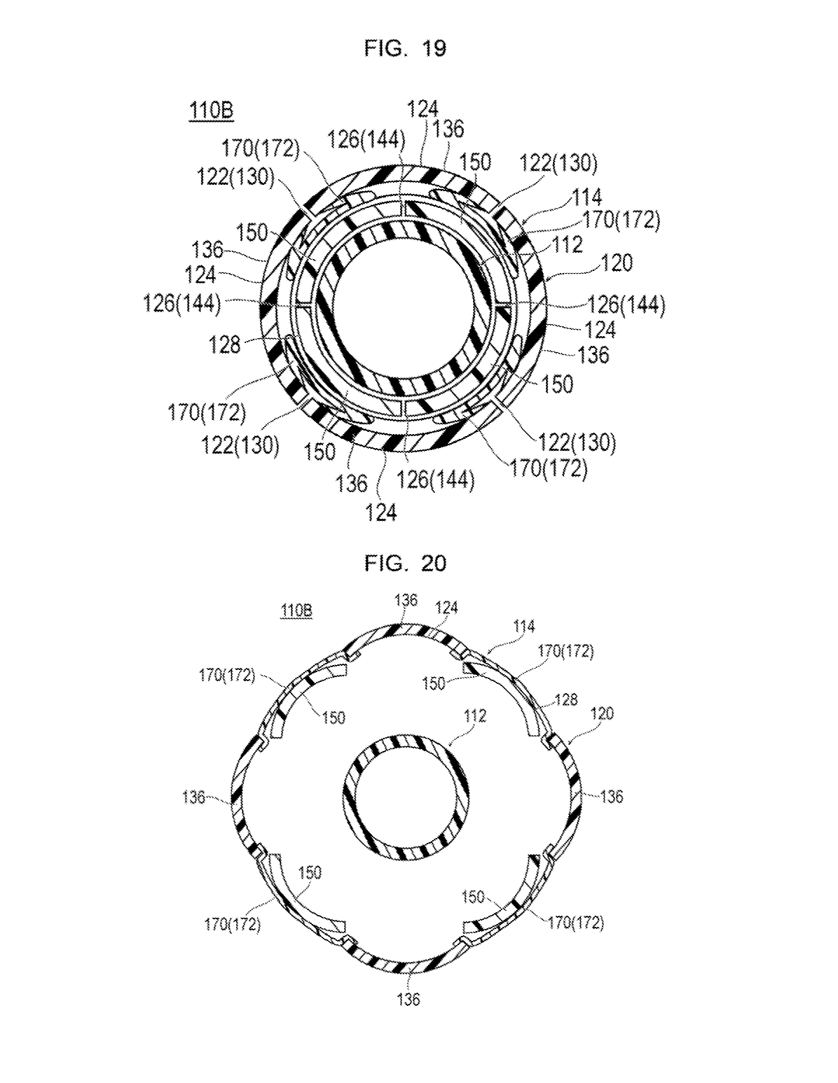

In an expansion device 110B according to a fourth embodiment of the present disclosure, as illustrated in FIGS. 19 and 20, the structure of the expansion mechanism 120 is different from that of the third embodiment. Note that, in the expansion device 110B according to the fourth embodiment, the same reference signs will be applied to elements exhibiting a function and an effect same as or similar to those of the expansion device 110A according to the third embodiment, and the detailed description will be omitted.

While the first expansion portion 136 is in an expansion state, the expansion mechanism 120 of the expansion device 110B has a restriction portion 170 that restricts the second expansion portion 150 protruding outward beyond the first expansion portion 136 by being disposed between the first expansion portions 136 adjacent to each other in the circumferential direction and outside the second expansion portion 150. In the embodiment, the restriction portion 170 is configured to be formed of a film member 172.

As illustrated in FIG. 19, while the expansion mechanism 120 is in a contraction state, a plurality of the film members 172 are disposed so as to be arranged in the circumferential direction corresponding to the plurality of first slits 122 formed with space therebetween in the circumferential direction. The film members 172 are folded by being interposed between the first expansion portion 136 and the second expansion portion 150. The film members 172 are bonded to the inner surfaces of edge portions of the first expansion portions 136 adjacent to each other in the circumferential direction. As illustrated in FIG. 20, the film members 172 are deployed in response to expansion of the space between the first expansion portions 136 adjacent to each other in the circumferential direction when the expansion mechanism 120 expands.

As the material configuring the film member 172, it is possible to exemplify polyolefin (for example, polyethylene, polypropylene, polybutene, an ethylene-propylene copolymer, an ethylene-vinyl acetate copolymer, an ionomer, and a mixture of two types or more thereof) and a polymer material such as polyvinyl chloride, polyamide, a polyamide elastomer, polyurethane, a polyurethane elastomer, polyimide, a fluorine resin, and a mixture thereof, or a multi-layer film formed of two types or more polymer materials.

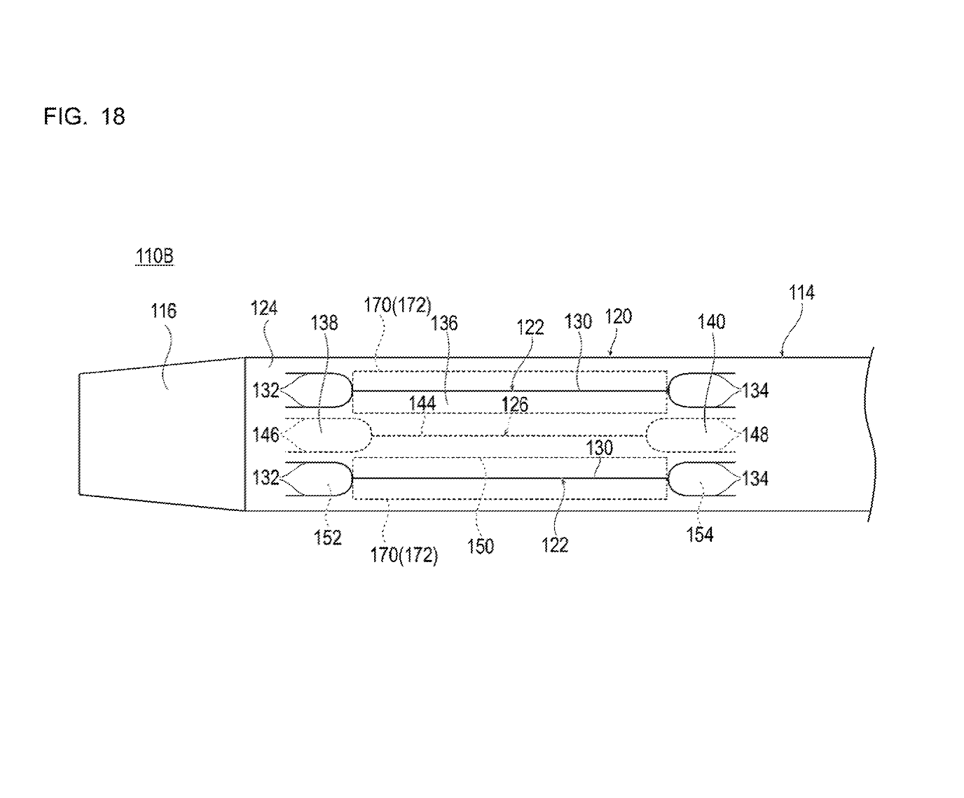

As illustrated in FIG. 18, the film member 172 may extend along the first slit 122. In this case, the film member 172 may be provided over a range throughout the substantially overall length of the main slit 130. Accordingly, while the expansion mechanism 120 is in an expansion state, the gap formed between the first expansion portions 136 adjacent to each other in the circumferential direction can be eliminated. Thus, the expansion target can be more uniformly widened in the circumferential direction.

Note that, in the present embodiment, the plurality of film members 172 are disposed in the circumferential direction. However, in place of such a configuration, a film body continuously surrounding the expansion mechanism 120 in the circumferential direction may be folded at multiple places in the circumferential direction so as to be similar to the film members 172 and may be bonded to the inner surfaces of the first expansion portions 136 at places similar to the film members 172. In other words, the plurality of film members 172 may be formed of one continuous film body.

According to the expansion device 110B having the above-described configuration, when the expansion mechanism 120 expands, due to an operation of the restriction portion 170, the second expansion portion 150 is prevented from protruding outward beyond the first expansion portion 136. Thus, the second expansion portion 150 can be prevented from failing to contract again.

In the case of the present embodiment, the restriction portion 170 is configured to be formed of the film member 172 which is folded while the expansion mechanism 120 is in a contraction state and is deployed in response to expansion of the space between the first expansion portions 136 adjacent to each other in the circumferential direction when the expansion mechanism 120 expands. Thus, the restriction portion 170 can be realized through a simple configuration.

In addition, since the film member 172 extends along the first slit 122, a gap between the first expansion portions 136 adjacent to each other in the circumferential direction is eliminated over a region elongated in the axial direction. Thus, the expansion target can be more uniformly widened in the circumferential direction.

In the case of the present embodiment, the film member 172 is interposed between the first expansion portion 136 and the second expansion portion 150 while the expansion mechanism 120 is in a contraction state. Thus, the film members 172 are folded and stored inside the expansion mechanism 120. Accordingly, the expansion mechanism 120 can be effectively suppressed from being increased in diameter due to the provided film member 172.

Note that, in the fourth embodiment, it is natural that each of the configuration portions common to those in the third embodiment can obtain an operation and an effect same as or similar to the operation and the effect achieved by each of the configuration portions common to those in the first embodiment.

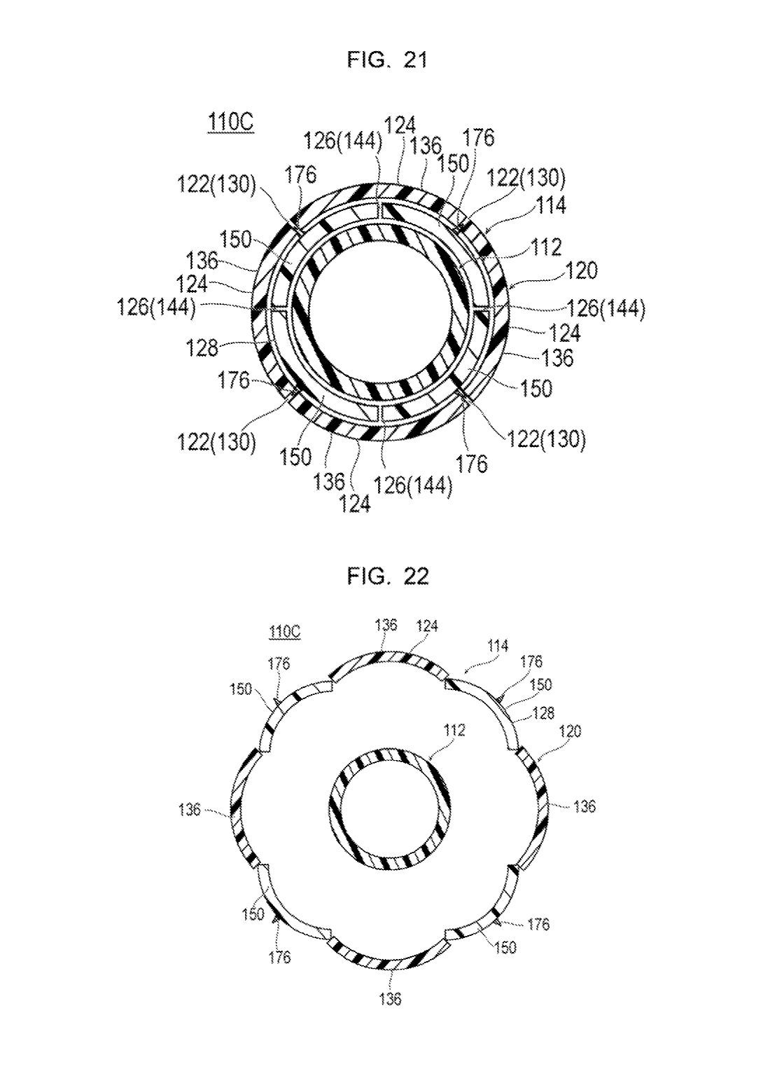

In an expansion device 110C according to a fifth embodiment of the present disclosure, as illustrated in FIGS. 21 and 22, the structure of the expansion mechanism 120 is different from that of the third embodiment. Note that, in the expansion device 110C according to the fifth embodiment, the same reference signs will be applied to elements exhibiting a function and an effect same as or similar to those of the expansion device 110A according to the third embodiment, and the detailed description will be omitted.

In the expansion device 110C, the expansion mechanism 120 has a plurality of wire-like cutting members 176 (blades) disposed on the outer surface of the second expansion portions 150 in the circumferential direction. In the present embodiment, one cutting member 176 is provided in one second expansion portion 150, and four cutting members 176 are provided with space among them in the circumferential direction. The cutting members 176 extend parallel to the axial line of the outer tube 114.

The cutting members 176 are elongated members for making a cut-off in a lesion area when the expansion mechanism 120 expands at the lesion area. For example, the cross-sectional shape of the cutting member 176 exhibits a shape which is tapered radially outward in the second pipe portion 128. In order to easily make a cut-off in a target lesion area, a sharp blade edge may be formed at the outer end of the cutting member 176. Note that, the sharp blade edge does not have to be provided.

The cutting member 176 is disposed at the position of the main slit 130 of the first slit 122. As in FIG. 21, while the expansion mechanism 120 is in a contraction state, the cutting member 176 does not protrude from the outer circumferential surface of the first pipe portion 124 and is not exposed to the outside of the outer tube 114. Meanwhile, as in FIG. 22, while the expansion mechanism 120 is in an expansion state, the cutting member 176 protrudes via a space between the first expansion portions 136 adjacent to each other in the circumferential direction and is exposed to the outside of the outer tube 114.

As the material configuring the cutting member 176, it is possible to exemplify metals (including an alloy), a resin, and the like. As such metals, it is possible to exemplify stainless steel, aluminum, an aluminum alloy, titanium, a titanium alloy, copper, a copper-based alloy, tantalum, and a cobalt alloy. As such a resin, it is possible to exemplify polyvinyl chloride, polyethylene, polypropylene, cyclic polyolefin, polystyrene, poly-(4-methylpentene-1), polycarbonate, an acrylic resin, an acrylonitrile-butadiene-styrene copolymer, polyester such as polyethylene terephthalate and polyethylene naphthalate, a butadiene-styrene copolymer, and polyamide (for example, nylon 6, nylon 6.6, and nylon 6.10, nylon 12).

In a case where treatment is performed by adopting the expansion device 110C having the above-described configuration, the expansion mechanism 120 is caused to arrive at a lesion area (stenosed site and the like) through a procedure similar to that of the above-described expansion device 110A. When the expansion mechanism 120 expands in a state where the expansion mechanism 120 is positioned at a position within the lesion area, the lesion area is widened, and thus, treatment of the lesion area can be performed. When the expansion mechanism 120 expands, the cutting members 176 provided on the outer surface of the second expansion portion 150 make a cut-off in the lesion area and the lesion area is caused to expand by the first expansion portion 136 and the second expansion portion 150. Thus, even a hardened lesion area (highly-calcified lesion) can be easily caused to expand.

Note that, in the expansion device 110C, similar to the expansion device 110B according to the fourth embodiment, the film members 172 may be provided. In this case, when the expansion mechanism 120 expands, the cutting members 176 break through the film members 172 and can make a cut-off in the lesion area.

In the fifth embodiment, it is natural that each of the configuration portions common to those in the third embodiment can obtain an operation and an effect same as or similar to the operation and the effect achieved by each of the configuration portions common to those in the third embodiment.

In a case where the above-described expansion devices 110A to 110C are used as a device which directly widens a lesion area by applying the expansion mechanism 120, at least one or both of the outer surfaces of the first expansion portion 136 and the second expansion portion 150 may be coated with a medicine. The entire outer surface of the first expansion portion 136 or the entire outer surface of the second expansion portion 150 may be coated with a medicine, and a part of the outer surface of the first expansion portion 136 or a part of the outer surface of the second expansion portion 150 may be coated with a medicine.

The medicine is a biological physiologically-active substance, which adheres to the lesion area when the expansion mechanism 120 causes the lesion area to expand. The medicine has an effect of suppressing restenosis by gradually flowing out to the expanded lesion area. As such a medicine, it is possible to exemplify an anti-cancer agent, an immunosuppressive agent, an antibiotic, an anti-rheumatic drug, an anti-thrombotic agent, an anti-hyperlipidemia drug, an ACE inhibitor, a calcium antagonist, an integrin inhibitor, an anti-allergic agent, an anti-oxidant, a GPIIbIIIa antagonist, retinoid, flavonoid, carotenoid, a lipid improver, a DNA synthesis inhibitor, a tyrosine kinase inhibitor, an antiplatelet agent, a vascular smooth muscle growth inhibitor, an anti-inflammatory agent, a living body-derived material, interferon, and a NO production promoting substance.

The above-described expansion devices 110A to 110C do not have to include the first distal side fragile portion 138, the first proximal side fragile portion 140, the second distal side fragile portion 152, and the second proximal side fragile portion 154.

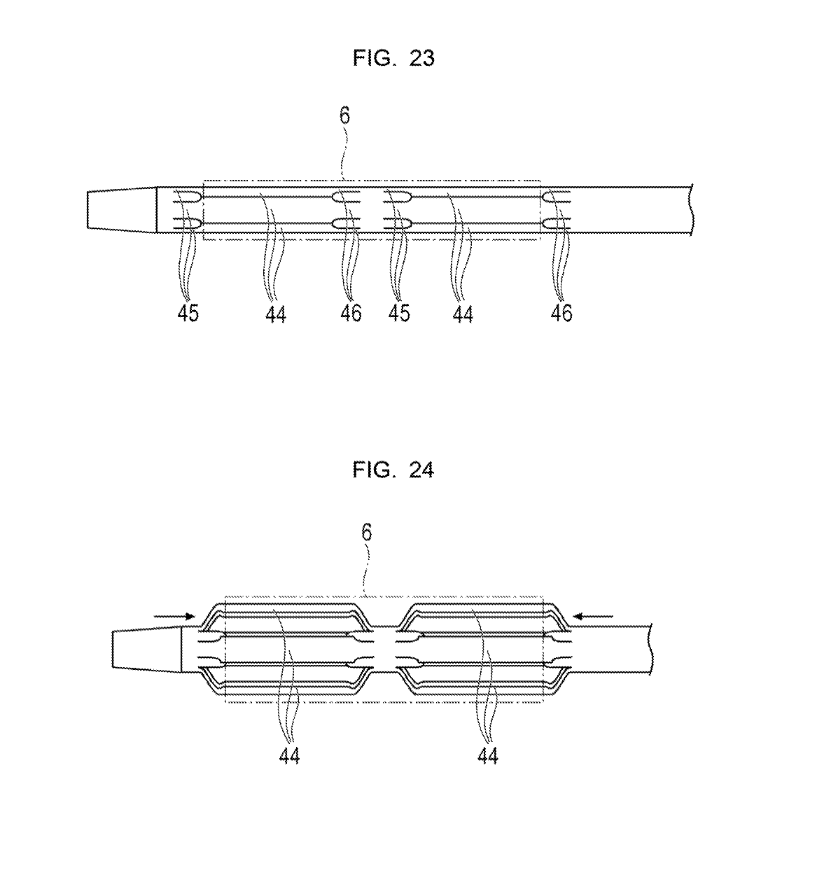

Note that, the present disclosure is not limited to only the above-described embodiments and various changes can be made by those skilled in the art within the technical concept of the present disclosure. For example, a plurality of the expansion mechanisms of the expansion device according to the first to fifth embodiments may be formed so as to be arranged in the axial direction. As an example, in the modification example illustrated in FIG. 23, the plurality of expansion portions 44 are formed so as to be arranged in the axial direction. According to such a configuration, as illustrated in FIG. 24, even the stent 6 elongated in the axial direction can favorably expand. In other words, when the expansion portion is excessively long, expansion force easily decreases at the central portion of the expansion portion. However, even the stent 6 elongated in the axial direction can favorably expand by providing the plurality of expansion portions 44.

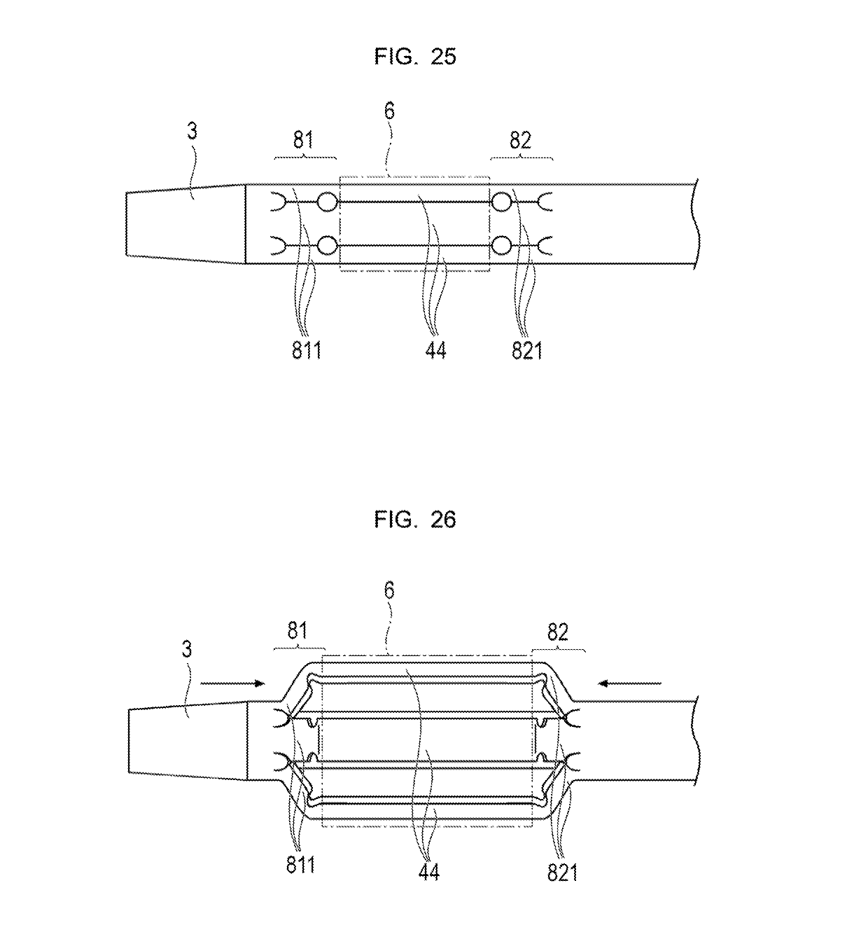

In addition, as illustrated in FIG. 25, portions 811 and 821 partially having high flexural rigidity may be provided in a distal side fragile portion 81 and a proximal side fragile portion 82 of the expansion device 1 according to the first embodiment. According to such a configuration, as illustrated in FIG. 26, high expansion force can be obtained by limiting the curved positions of the distal side fragile portion 81 and the proximal side fragile portion 82 to positions excluding the positions of the portions 811 and 821 having high rigidity, in the distal side fragile portion 81 and the proximal side fragile portion 82.

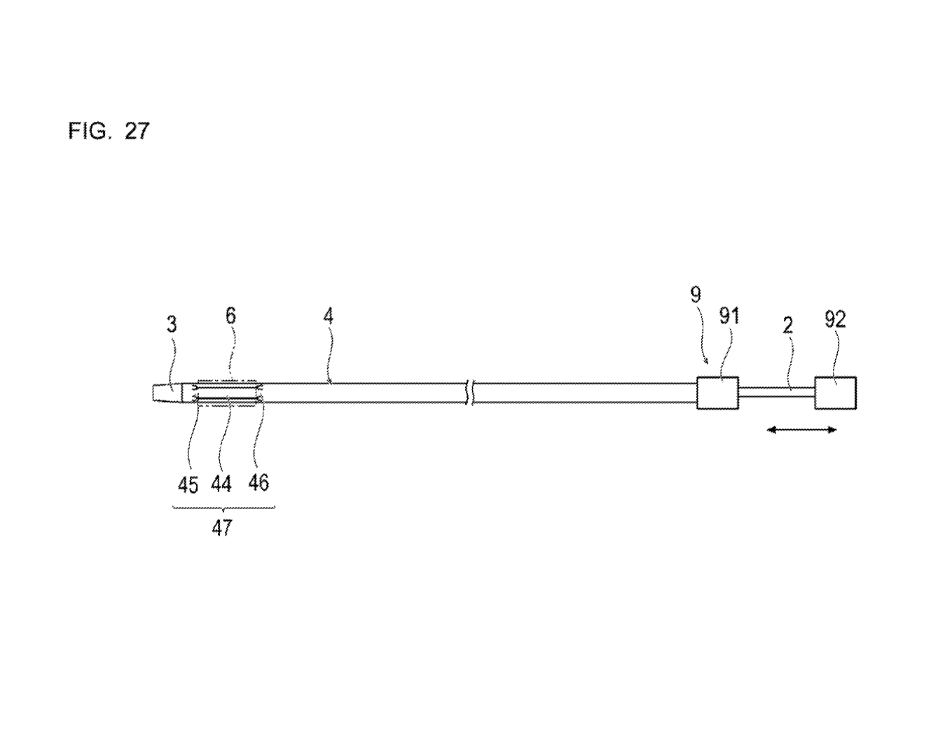

In addition, as illustrated in FIG. 27, an operation unit 9 for operating the inner tube 2 and the outer tube 4 of the expansion device 1 according to the first embodiment may have a structure in which a first hub 91 fixedly attached to the proximal portion of the outer tube 4 and a second hub 92 fixedly attached to the proximal portion of the inner tube 2 introduced through the proximal side opening portion of the outer tube 4 can relatively move. In addition, the operation unit of the expansion devices 110A to 110C according to the third to fifth embodiments may have a configuration similar to that of the operation unit 71 of the second embodiment illustrated in FIG. 10 or that of the operation unit 9 illustrated in FIG. 27.

In addition, the inner tube 2 (core) does not have to be the pipe body as long as compressive force can act on the outer tube 4.

In addition, in each of the above-described embodiments, a stent is installed in the expansion portion. However, without installing the stent, the expansion portion may be configured to directly press a biological lumen. In addition, the present disclosure can be applied to a medical device as long as the medical device is provided with the expansion portion. For example, the present disclosure may be applied to a catheter used in place of a cutting balloon catheter in which a blade is provided on the surface of the balloon. In this case, the expansion portion (linear portion) can play a role of the balloon, and the expansion portion itself can function as the blade. However, a separate blade may be attached to the expansion portion. In addition, for example, the present disclosure may be applied to a foreign body collecting catheter in which a net-like portion for collecting a foreign body is provided. In this case, the expansion portion (linear portion) itself can function as a net collecting a foreign body. However, a separate net structure may be attached to the expansion portion.

In addition, the outer tube may be formed in advance such that a portion intended to be curved is curved in a desired direction.

In addition, the configurations of the above-described embodiments can be appropriately combined together.

The detailed description above describes an expansion device. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An expansion device comprising:
a pipe body in which a plurality of wire portions extending along an axial direction are disposed so as to be arranged in a circumferential direction in a distal portion; and
a core that is inserted into the pipe body and is interlocked with the distal portion of the pipe body,
wherein the wire portion has an expansion portion which expands radially outward when the pipe body is compressed in the axial direction, a distal side fragile portion which is formed on a distal side of the expansion portion and has flexural rigidity lower than that of the expansion portion, and a proximal side fragile portion which is formed on a proximal side of the expansion portion and has flexural rigidity lower than that of the expansion portion.

2. The expansion device according to claim 1,
wherein the distal side fragile portion and the proximal side fragile portion are set to have flexural rigidity lower than that of the expansion portion due to at least one of a material, a width in the circumferential direction, and a thickness different from that of the expansion portion.

3. The expansion device according to claim 1,
wherein in the pipe body, a main slit is formed between a plurality of expansion portions adjacent to each other in the circumferential direction, a plurality of bifurcated slits bifurcated from at least one of the distal side and the proximal side of the main slit are formed, and a width of at least one of the distal side fragile portion and the proximal side fragile portion in the circumferential direction is set to be smaller than that of the expansion portion due to the provided bifurcated slits.

4. The expansion device according to claim 1,
wherein the pipe body has a first pipe portion in which a plurality of first slits are formed with space therebetween in the circumferential direction, and a second pipe portion which is disposed between the first pipe portion and the core and in which a plurality of second slits are formed with space therebetween in the circumferential direction,
wherein the expansion portion include a plurality of first expansion portions which are formed between the first slits in the first pipe portion, and a plurality of second expansion portions which are formed between the second slits in the second pipe portion, and
wherein the plurality of first slits and the plurality of second slits are formed at positions different from each other in the circumferential direction.

5. The expansion device according to claim 4,
wherein the plurality of first expansion portions and the plurality of second expansion portions are alternately arranged in the circumferential direction while the expansion portion is in an expansion state.

6. The expansion device according to claim 5, further comprising:
a restriction portion that restricts the plurality of second expansion portions from protruding outward beyond the plurality of first expansion portions by being disposed between the plurality of first expansion portions adjacent to each other in the circumferential direction and outside the plurality of second expansion portions while the plurality of first expansion portions are in an expansion state.

7. The expansion device according to claim 6,
wherein the restriction portion is a film member which is folded while the expansion portion is in a contraction state and is deployed in response to expansion of the space between the plurality of first expansion portions adjacent to each other in the circumferential direction when the expansion portion expands.

8. The expansion device according to claim 7,
wherein the film member extends along the first slit.

9. The expansion device according to claim 8,
wherein the film member is interposed between the plurality of first expansion portions and the plurality of second expansion portions while the expansion portion is in a contraction state.

10. The expansion device according to claim 4,
wherein the first pipe portion has a plurality of first wire portions which are disposed so as to be arranged in the circumferential direction,
wherein the second pipe portion has a plurality of second wire portions which are disposed so as to be arranged in the circumferential direction,
wherein the first wire portion has the plurality of first expansion portions which expand radially outward when the first pipe portion is compressed in the axial direction, a first distal side fragile portion which is formed on the distal side of the plurality of first expansion portions and has flexural rigidity lower than that of the plurality of first expansion portions, and a first proximal side fragile portion which is formed on the proximal side of the plurality of first expansion portions and has flexural rigidity lower than that of the plurality of first expansion portions, and
wherein the second wire portion has the plurality of second expansion portions which expand radially outward when the second pipe portion is compressed in the axial direction, a second distal side fragile portion which is formed on the distal side of the plurality of second expansion portions and has flexural rigidity lower than that of the plurality of second expansion portions, and a second proximal side fragile portion which is formed on the proximal side of the plurality of second expansion portions and has flexural rigidity lower than that of the plurality of second expansion portions.

11. The expansion device according to claim 10,
wherein the second distal side fragile portion has a length in the axial direction longer than that of the first distal side fragile portion, and
wherein the second proximal side fragile portion has a length in the axial direction longer than that of the first proximal side fragile portion.

12. The expansion device according to claim 1,
wherein the expansion portion includes an X-ray contrast material.

13. The expansion device according to claim 1,
wherein the core includes an X-ray contrast material in a portion positioned on an inner side of the expansion portion.

14. The expansion device according to claim 4,
wherein the core includes an X-ray contrast material in a portion positioned on an inner side of the expansion portion.

15. An expansion device comprising;
a pipe body in which a plurality of wire portions extending along an axial direction are disposed so as to be arranged in a circumferential direction in a distal portion;
a core that is inserted into the pipe body and is interlocked with the distal portion of the pipe body;
wherein the wire portion has an expansion portion which expands radially outward when the pipe body is compressed in the axial direction, a distal side fragile portion which is formed on a distal side of the expansion portion and has flexural rigidity lower than that of the expansion portion, and a proximal side fragile portion which is formed on a proximal side of the expansion portion and has flexural rigidity lower than that of the expansion portion, wherein the distal side fragile portion and the proximal side fragile portion are set to have flexural rigidity lower than that of the expansion portion due to at least one of a material, a width in the circumferential direction, and a thickness different from that of the expansion portion; and
wherein in the pipe body, a main slit is formed between a plurality of expansion portions adjacent to each other in the circumferential direction, a plurality of bifurcated slits bifurcated from at least one of the distal side and the proximal side of the main slit are formed, and a width of at least one of the distal side fragile portion and the proximal side fragile portion in the circumferential direction is set to be smaller than that of the expansion portion due to the provided bifurcated slits.

16. The expansion device according to claim 15,
wherein the expansion portion includes an X-ray contrast material.

17. The expansion device according to claim 15,
wherein the core includes an X-ray contrast material in a portion positioned on an inner side of the expansion portion.

18. A method of treating a blood vessel, the method comprising:
positioning an expansion member within the blood vessel, the expansion member including a pipe body in which a plurality of wire portions extending along an axial direction are disposed so as to be arranged in a circumferential direction in a distal portion, and a core that is inserted into the pipe body and is interlocked with the distal portion of the pipe body, wherein the wire portion has an expansion portion which expands radially outward when the pipe body is compressed in the axial direction, a distal side fragile portion which is formed on a distal side of the expansion portion and has flexural rigidity lower than that of the expansion portion, and a proximal side fragile portion which is formed on a proximal side of the expansion portion and has flexural rigidity lower than that of the expansion portion; and
radially expanding the expansion portion within the blood vessel.

19. The method according to claim 18, comprising:
placing a stent on an outer circumferential surface of the expansion portion of the expansion member before inserting the expansion member into a stenosed site of the blood vessel; and
radially expanding the expansion portion and the stent into the stenosed site within the blood vessel and indwelling the stent within the stenosed site.

20. The method according to claim 18, comprising:
setting the distal side fragile portion and the proximal side fragile portion to have flexural rigidity lower than that of the expansion portion due to at least one of a material, a width in the circumferential direction, and a thickness different from that of the expansion portion.
